(19) United States
(12) Patent Application Publication
(10) Pub. No.: US 2023/0373898 A1
(43) Pub. Date: Nov. 23, 2023

(54) METHOD FOR PRODUCING CARBONYL HALIDE

(71) Applicants: NATIONAL UNIVERSITY CORPORATION KOBE UNIVERSITY, Kobe-shi (JP); AGC Inc., Tokyo (JP)

(72) Inventors: Akihiko TSUDA, Kobe-shi (JP); Takashi OKAZOE, Tokyo (JP)

(73) Assignees: NATIONAL UNIVERSITY CORPORATION KOBE UNIVERSITY, Kobe-shi (JP); AGC Inc., Tokyo (JP)

(21) Appl. No.: 18/365,276

(22) Filed: Aug. 4, 2023

Related U.S. Application Data
(63) Continuation of application No. PCT/JP2022/002662, filed on Jan. 25, 2022.

(30) Foreign Application Priority Data
Feb. 12, 2021 (JP) 2021-021002

(51) Int. Cl. C07C 51/58

(52) U.S. Cl. CPC C07C 51/58

(57) ABSTRACT

The objective of the present invention is to provide a method for producing a carbonyl halide efficiently to the used halogenated hydrocarbon. The method for producing a carbonyl halide according to the present invention is characterized in comprising the steps of preparing a mixed gas comprising oxygen and a C₂₋₄ halogenated hydrocarbon having one or more halogeno groups selected from the group consisting of chloro, bromo and iodo, and flowing the mixed gas and irradiating a high energy light to the flowed mixed gas.

### TECHNICAL FIELD

The present invention relates to a method for producing a carbonyl halide efficiently in terms of an amount of a used halogenated hydrocarbon.

### BACKGROUND ART

A carbonyl halide such as phosgene is very important as a synthetic intermediate of various compounds and a raw material of a material. For example, a carbonate compound is generally produced from phosgene and an alcohol compound.

Phosgene is however very toxic. For example, phosgene is easily reacted with water to generate hydrogen chloride and has a history of being used as poisonous gas. In general, phosgene is produced by a high-heat-generating gas phase reaction between an anhydrous chlorine gas and high purity carbon monoxide in the presence of an activated carbon catalyst. Carbon monoxide used in this reaction is also toxic. The basic process to produce phosgene has not changed much since the 1920s. Such a process to produce phosgene requires expensive large-scale facilities. An extensive safety assurance is essential in plant design due to the high toxicity of phosgene and leads to increased production costs.

The inventors of the present invention have developed the method for producing a halogen and/or a carbonyl halide by irradiating light to a halogenated hydrocarbon in the presence of oxygen (Patent document 1). The method is safe, since the carbonyl halide produced by the method can be directly supplied to an amine compound and an alcohol compound to be reacted. In addition, the carbonyl halide which has not used for the reaction can be collected not to be leaked outside by using a trap. For example, the inventors also have developed the method for producing a halogenated carboxylic acid ester by irradiating light to a mixture containing a halogenated hydrocarbon and an alcohol in the presence of oxygen (Patent document 2). In addition, the inventors also have developed the method for producing a carbonate derivative by irradiating light to a composition containing a halogenated hydrocarbon, a nucleophilic functional group-containing compound and a base in the presence of oxygen (Patent document 3 and Patent document 4).

When a carbonyl halide is produced by the above-described method, a large amount of a halogenated hydrocarbon remains in comparison of the produced carbonyl halide. A halogenated hydrocarbon cannot be easily discarded and is needed to be purified and reused due to high environmental load.

It has been known from a long time ago that a carbonyl halide is decomposed by light. For example, Patent document 5 discloses the method for decomposing phosgene to be removed by photodecomposition by irradiating ultraviolet light to boron trichloride containing phosgene as an impurity. It is also described in Non-patent document 1 that phosgene is decomposed by irradiating light.

### PRIOR ART DOCUMENT

### Patent Document

Patent document 1: JP 2013-181028 A
Patent document 2: WO 2015/156245
Patent document 3: WO 2018/211952
Patent document 4: WO 2018/211953
Patent document 5: U.S. Pat. No. 4,405,423 B

### Non-Patent Document

Non-patent document 1: C. W. Mostgomery, et al., J. Am. Chem. Soc., 1934, 56, 5, pp. 1089-1092

### DISCLOSURE OF THE INVENTION

### Problems to be Solved by the Invention

The inventors of the present invention have developed a method for producing a carbonyl halide by irradiating light to a halogenated hydrocarbon as described above, but the yield to the used halogenated hydrocarbon is low.

Thus, the objective of the present invention is to provide a method for producing a carbonyl halide efficiently to the used halogenated hydrocarbon.

### Means for Solving the Problems

The inventors of the present invention repeated intensive studies in order to solve the above-described problems. For example, the inventors anticipated that if high energy light is irradiated to vaporized halogenated hydrocarbon, the halogenated hydrocarbon can be efficiently photodecomposed but the produced carbonyl halide may be rapidly photodecomposed in a gas phase. On the one hand, the inventors examined various reaction conditions; as a result, the inventors completed the present invention by finding that a carbonyl halide can be surprisingly produced with high yield by irradiating high energy light to a vaporized flowed halogenated hydrocarbon.

The present invention is hereinafter described.

[1] A method for producing a carbonyl halide, comprising the steps of:
preparing a mixed gas comprising oxygen and a C₂₋₄ halogenated hydrocarbon having one or more halogeno groups selected from the group consisting of chloro, bromo and iodo, and
flowing the mixed gas and irradiating a high energy light to the flowed mixed gas.

[2] The method according to the above [1], wherein a shortest distance from a light source of the high energy light to the flowed mixed gas is 1 m or less.

[3] The method according to the above [1] or [2], wherein time to irradiate the high energy light to the flowed mixed gas is 1 second or more and 10000 seconds or less.

[4] The method according to any one of the above [1] to [3], wherein a temperature during the irradiation of the high energy light to the flowed mixed gas is 120° C. or higher and 300° C. or lower.

[5] A method for producing a carbonate compound, comprising the steps of:
producing a halogenated carbony by the method according to any one of the above [1] to [4], and
reacting an alcohol compound and the carbonyl halide,
wherein a molar ratio of the alcohol compound to the C₂₋₄ halogenated hydrocarbon is adjusted to 1 or more.

[6] A method for producing a halogenated formic acid ester compound, comprising the steps of:
producing a halogenated carbony by the method according to any one of the above [1] to [4], and
reacting an alcohol compound and the carbonyl halide,
wherein a molar ratio of the alcohol compound to the C₂₋₄ halogenated hydrocarbon is adjusted to less than 1.

[7] A method for producing an isocyanate compound, comprising the steps of:
producing a halogenated carbony by the method according to any one of the above [1] to [4], and
reacting a primary amine compound and the carbonyl halide,
wherein a molar ratio of the primary amine compound to the C₂₋₄ halogenated hydrocarbon is adjusted to less than 1.

[8] A method for producing an amino acid-N-carboxylic anhydride, comprising the steps of:
producing a halogenated carbony by the method according to any one of the above [1] to [4], and
reacting an amino acid compound represented by the following formula (VII) and the carbonyl halide,
wherein the amino acid-N-carboxylic anhydride is represented by the following formula (VIII):

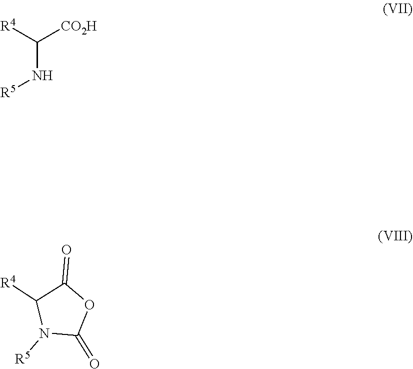

wherein
R⁴ is an amino acid side chain group wherein a reactive group is protected,
R⁵ is H or P¹—[—NH—CHR⁶—C(═O)—]₁— wherein R⁶ is an amino acid side chain group wherein a reactive group is protected, P¹ is a protective group of the amino group, 1 is an integer of 1 or more, and when 1 is an integer of 2 or more, a plurality of R⁶ may be the same as or different from each other.

[9] A method for producing a Vilsmeier reagent,
wherein the Vilsmeier reagent is a salt represented by the following formula (X):

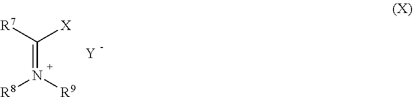

wherein
R⁷ is a hydrogen atom, a C₁₋₆ alkyl group or an optionally substituted C₆₋₁₂ aromatic hydrocarbon group,
R⁸ and R⁹ are independently a C₁₋₆ alkyl group or an optionally substituted C₆₋₁₂ aromatic hydrocarbon group, or R⁸ and R⁹ may form a 4 or more and 7 or less-membered ring structure together with each other,
X is a halogeno group selected from the group consisting of chloro, bromo and iodo,
Y⁻ is a counter anion,
comprising the steps of:
producing a halogenated carbony by the method according to any one of the above [1] to [4], and
reacting the carbonyl halide and an amino acid compound represented by the following formula (IX):

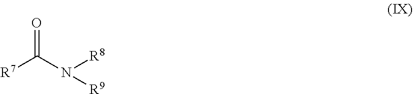

wherein R⁷ to R⁹ have the same meanings as the above.

### Effect of the Invention

The use and treatment of a halogenated hydrocarbon is restricted due to a high environmental impact. On the one hand, a carbonyl halide can be produced efficiently to the used halogenated hydrocarbon and can be effectively used by the present invention. Thus, the present invention is industrially useful as the technology that enable effective utilization of a halogenated hydrocarbon and efficient production of a carbonyl halide such as phosgene.

### BRIEF DESCRIPTION OF THE DRAWINGS

FIG. 1 is a schematic diagram to demonstrate one example of the constitution of a reaction apparatus usable in the present invention method.

FIG. 2 is a schematic diagram to demonstrate one example of the constitution of a reaction apparatus usable in the present invention method.

FIG. 3 is a schematic diagram to demonstrate one example of the constitution of a reaction apparatus usable in the present invention method.

FIG. 4 is a schematic diagram to demonstrate one example of the constitution of a reaction apparatus usable in the present invention method.

FIG. 5 is a schematic diagram to demonstrate one example of the constitution of a reaction apparatus usable in the present invention method.

FIG. 6 is a schematic diagram to demonstrate one example of the constitution of a reaction apparatus usable in the present invention method.

### MODE FOR CARRYING OUT THE INVENTION

The present invention method is hereinafter described step by step, and the present invention is not restricted to the following specific examples.

### 1. Step to Prepare Mixed Gas

The mixed gas comprising oxygen and the C₂₋₄ halogenated hydrocarbon comprising one or more halogeno groups selected from the group consisting of chloro, bromo and iodo is prepared in this step.

The C₂₋₄ halogenated hydrocarbon used in this step means a hydrocarbon that has the carbon number of 2 or more and 4 or less and has one or more halogeno groups selected from the group consisting of chloro, bromo and iodo. The C₂₋₄ halogenated hydrocarbon may be decomposed by oxygen and high energy light to become a carbonyl halide or a carbonyl halide-like compound.

The C₂₋₄ halogenated hydrocarbon may be decomposed by oxygen and high energy light as described above to produce a carbonyl halide or a carbonyl halide-like compound in the present invention. The carbonyl halide-like compound may play a role similarly to the carbonyl halide. The C₂₋₄ halogenated hydrocarbon is preferably a C₂ halogenated hydrocarbon and more preferably a halogenated ethane and a halogenated ethene. The C₂₋₄ halogenated hydrocarbon is preferably an alkene or an alkyne having one or more unsaturated bonds in order to be decomposed more easily. The C₂₋₄ halogenated hydrocarbon is more preferably a C₂₋₄ polyhalogenated hydrocarbon, preferably a C₂₋₄ perhalogenated hydrocarbon, of which all hydrogen atoms are substituted with halogeno groups, and more preferably a C₂ perhalogenated hydrocarbon. In addition, a halogenated hydrocarbon having two or more halogeno groups on one carbon atom is preferred, though the halogeno group may be transferred by the decomposition. Furthermore, a C₂₋₄ chloro hydrocarbon is preferred, a C₂₋₃ chloro hydrocarbon is more preferred and a C₂ chloro hydrocarbon is even more preferred in terms of the easy vaporization.

An example of the specific C₂₋₄ halogenated hydrocarbon includes a halogenated ethane such as 1,1,2-trichloroethane, 1,1,1-trichloroethane, 1,1,2,2-tetrachloroethane and 1,1,1,2-tetrachloroethane; a halogenated ethene such as 1,1,2-trichloroethene and 1,1,2-tribromoethene; a halogenated propane such as 1,1,1,3-tetrachloropropane; a perhalogenated alkane such as hexachloroethane and hexabromoethane; and a perhalogenated ethene such as tetrachloroethylene and tetrabromoethylene.

The C₂₋₄ halogenated hydrocarbon may be appropriately selected depending on the target chemical reaction and the desired product. Only one kind of the C₂₋₄ halogenated hydrocarbon may be used, or two or more kinds of the C₂₋₄ halogenated hydrocarbons may be used in combination. Only one C₂₋₄ halogenated hydrocarbon is preferably used depending on the target compound to be produced. The C₂₋₄ halogenated hydrocarbon having a chloro group is preferred among the C₂₋₄ halogenated hydrocarbons in terms of vaporization and cost.

A general C₂₋₄ halogenated hydrocarbon product may contain a stabilizer such as an alcohol and an amine to inhibit the decomposition of the C₂₋₄ halogenated hydrocarbon product in some cases. Since the C₂₋₄ halogenated hydrocarbon product is oxidatively photodecomposed in the present invention, the C₂₋₄ halogenated hydrocarbon product from which a stabilizer is removed may be used. When the C₂₋₄ halogenated hydrocarbon product from which a stabilizer is removed is used, the C₂₋₄ halogenated hydrocarbon product may be possibly decomposed more efficiently. For example, high energy light of which energy is relatively low may be used, and the time to irradiate high energy light may be reduced. A method for removing a stabilizer from the C₂₋₄ halogenated hydrocarbon product is not particularly restricted. For example, the C₂₋₄ halogenated hydrocarbon product may be washed using water and then dried.

Tetrachloroethylene, i.e. Cl₂C═CCl₂, can be preferably used as the C₂₋₄ halogenated hydrocarbon used in the present invention method. Tetrachloroethylene is industrially used for washing chemical fiber and metal and is obtained as a side product during other chemical synthesis processes. For example, the C₂₋₄ halogenated hydrocarbon that has been once used as a solvent may be recovered to be reused. It is preferred that such a used C₂₋₄ halogenated hydrocarbon is purified to some extent for use, since if a large amount of an impurity and water is contained, the reaction may be possibly inhibited. For example, it is preferred that a water-soluble impurity is removed by washing with water and then the C₂₋₄ halogenated hydrocarbon is dried over anhydrous sodium sulfate, anhydrous magnesium sulfate or the like. An excessive purification by which the productivity becomes less is not needed, since even when about 1 mass % of water is contained, the reaction may proceed. The water content is preferably 0.5 mass % or less, more preferably 0.2 mass % or less, and even preferably 0.1 mass % or less. The water content is preferably detection limit or less, or 0 mass %. The above-described reused C₂₋₄ halogenated hydrocarbon may contain the degradant of the C₂₋₄ halogenated hydrocarbon or the like.

In particular, when the C₂₋₄ halogenated hydrocarbon is not liquid in an atmospheric temperature and an atmospheric pressure, a solvent may be used in addition to the C₂₋₄ halogenated hydrocarbon. Such a solvent may possibly accelerate the decomposition of the C₂₋₄ halogenated hydrocarbon. In addition, the solvent may possibly inhibit the decomposition of the carbonyl halide or the carbonyl halide-like compound produced by the oxidative decomposition of the C₂₋₄ halogenated hydrocarbon. It is preferred that the solvent can appropriately dissolve the C₂₋₄ halogenated hydrocarbon and does not inhibit the decomposition of the C₂₋₄ halogenated hydrocarbon. An example of the solvent includes a ketone solvent such as acetone, methyl ethyl ketone, methyl isobutyl ketone and cyclohexanone; an ester solvent such as ethyl acetate; an aliphatic hydrocarbon solvent such as n-hexane; an aromatic hydrocarbon solvent such as benzene, toluene, xylene and benzonitrile; an ether solvent such as diethyl ether, tetrahydrofuran and dioxane; and a nitrile solvent such as acetonitrile.

When the solvent is used in combination, the usage amount of the solvent may be appropriately adjusted. For example, the ratio of the C₂₋₄ halogenated hydrocarbon to the total of the C₂₋₄ halogenated hydrocarbon and the solvent may be adjusted to 50 mass % or more. The ratio is preferably 60 mass % or more or 70 mass % or more and more preferably 80 mass % or more or 90 mass % or more.

An oxygen source may be a gas containing oxygen, and for example, air or purified oxygen may be used. Purified oxygen may be mixed with an inert gas such as nitrogen and argon to be used. It is preferred to use air in terms of cost and easiness. An oxygen content in the oxygen-containing gas used as an oxygen source is preferably about 15 vol % or more and about 100 vol % or less in terms of high decomposition efficiency of the C₂₋₄ halogenated hydrocarbon by the irradiation of high energy light. Substantially oxygen only other than an inevitable impurity is preferably used. The oxygen content may be appropriately determined depending on the kind of the C₂₋₄ halogenated hydrocarbon or the like. For example, when a chloro hydrocarbon such as tetrachloroethylene is used as the C₂₋₄ halogenated hydrocarbon, the oxygen content is preferably 15 vol % or more and 100 vol % or less. When a bromo hydrocarbon is used, the oxygen content is preferably 90 vol % or more and 100 vol % or less. Even when oxygen having an oxygen content of 100 vol % is used, the oxygen content can be controlled in the above-described range by adjusting a flow rate of oxygen into the reaction system. Dried air may be used as the oxygen source. Since even air containing water vapor may not excessively inhibit the reaction, air may be used without adjusting water vapor content. The oxygen concentration in the air is about 21 vol %, and the oxygen concentration in the oxygen source may be also adjusted to 20±5 vol %. The concentration is preferably 20±2 vol %. When air is used as the oxygen source, an air component other than oxygen may absorb excessive high energy light and reduces the concentration of the produced carbonyl halide; as a result, the decomposition of the produced carbonyl halide may possibly be inhibited.

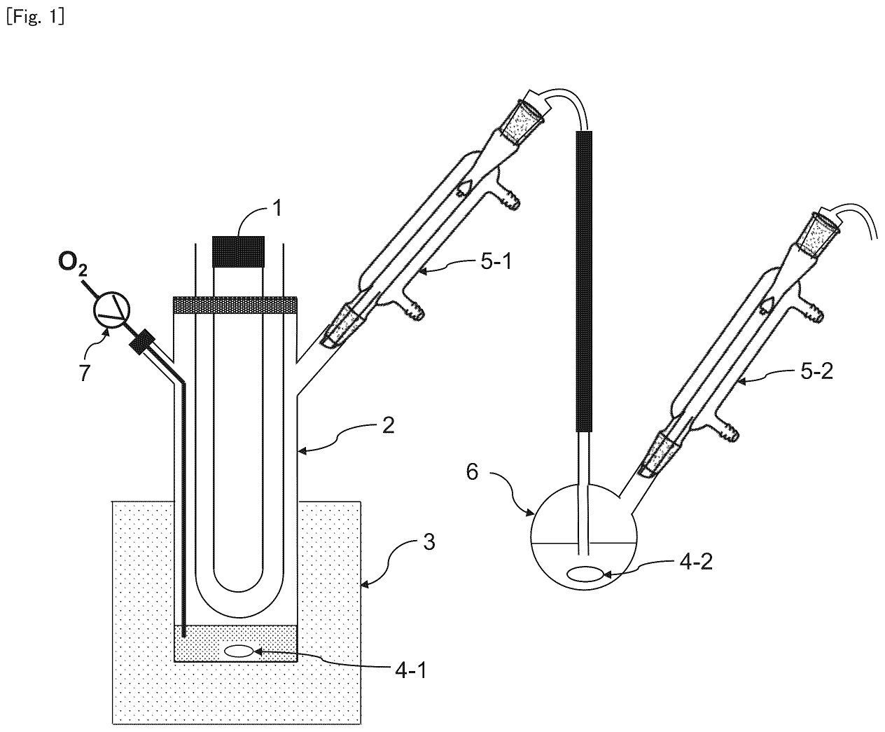

A mixed gas containing the gaseous C₂₋₄ halogenated hydrocarbon and oxygen is prepared in this step. The conditions to prepare the mixed gas are not particularly restricted. For example, the bath temperature of the photoreaction vessel 2 equipped with the light source 1 and the bath 3 is preliminarily adjusted to the boiling point or higher of the C₂₋₄ halogenated hydrocarbon and then the C₂₋₄ halogenated hydrocarbon is supplied into the photoreaction vessel 2 to be vaporized as demonstrated in FIG. 1. The supplied C₂₋₄ halogenated hydrocarbon may be stirred using the stirring bar 4 to accelerate the vaporization of the C₂₋₄ halogenated hydrocarbon. The C₂₋₄ halogenated hydrocarbon is vaporized and an oxygen-containing gas is supplied into the gas phase of the photoreaction vessel 2 at the predetermined flow rate to prepare the mixed gas containing the C₂₋₄ halogenated hydrocarbon and oxygen in the photoreaction vessel 2.

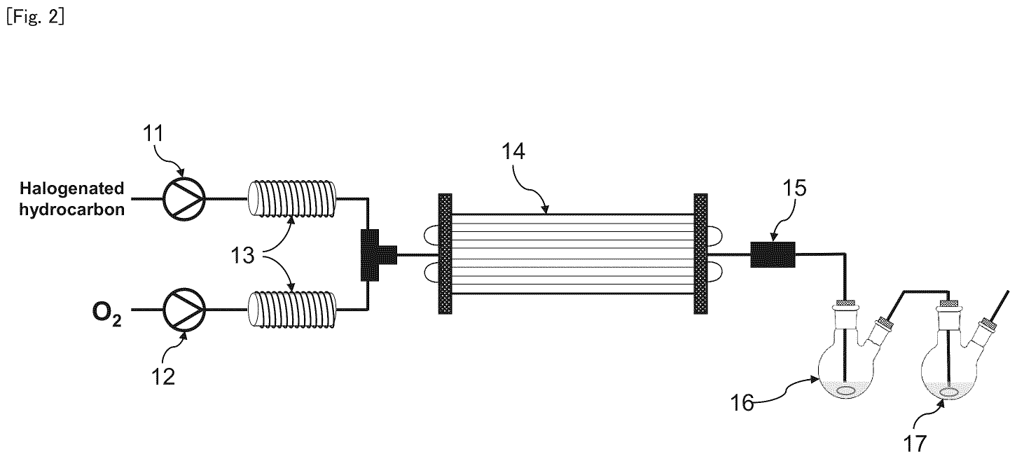

Alternatively, the C₂₋₄ halogenated hydrocarbon is supplied to the heater 13 at the predetermined flow rate using the syringe pump 11 and vaporized by heating to the boiling point or higher. The flow rate of an oxygen-containing gas is adjusted using the mass flow controller 12, and the oxygen-containing gas is mixed with the vaporized C₂₋₄ halogenated hydrocarbon to prepare the mixed gas as demonstrated in FIGS. 2 to 6.

The ratio of the vaporized C₂₋₄ halogenated hydrocarbon and oxygen in the mixed gas may be appropriately adjusted as long as a carbonyl halide can be successfully produced. For example, when the C₂₋₄ halogenated hydrocarbon and oxygen are mixed by flowing them, the ratio of the flow rate of oxygen in an oxygen-containing gas to the flow rate of the C₂₋₄ halogenated hydrocarbon in the mixed gas may be adjusted to 0.1 or more and 10 or less. When the ratio is 0.1 or more, the C₂₋₄ halogenated hydrocarbon may be oxidatively photodecomposed sufficiently. When the ratio is 10 or less, the produced carbonyl halide may be sufficiently prevented from further being oxidatively photodecomposed. The ratio is preferably 0.2 or more, more preferably 0.4 or more, even more preferably 0.5 or more, and preferably 8 or less, more preferably 6 or less. In particular, when the ratio is 0.5 or more, the production of a by-product and trouble of the reaction system due to a by-product may be suppressed more effectively.

When an oxygen-containing gas is supplied into the gas phase containing the vaporized C₂₋₄ halogenated hydrocarbon, an enough amount of oxygen to oxidatively photodecompose the C₂₋₄ halogenated hydrocarbon is preferably used. For example, the flow rate of oxygen per 1 minute to 1 mole of the C₂₋₄ halogenated hydrocarbon may be adjusted to 0.1 L or more and 100 L or less. The ratio is preferably 1 L or more, more preferably 5 L or more, and even more preferably 10 L or more.

2. Oxidative Photodecomposition Step

The mixed gas containing the C₂₋₄ halogenated hydrocarbon and oxygen is flowed, and high energy light is irradiated to the flowed mixed gas in the gas phase to produce a carbonyl halide by oxidatively photodecomposing the C₂₋₄ halogenated hydrocarbon in this step.

The high energy light to be irradiated to the flowed mixed gas preferably contains short wavelength light and more preferably contains ultraviolet light. The light containing the light having the wavelength of 180 nm or more and 500 nm or less and the light having the peak wavelength of 180 nm or more and 500 nm or less are preferred. The wavelength of the high energy light may be appropriately determined, is more preferably 400 nm or less and even more preferably 300 nm or less, and the light of which peak wavelength is included in the above ranges is also preferred. When the irradiated light contains the light of which wavelength is included in the above ranges, the C₂₋₄ halogenated hydrocarbon can be oxidatively photodecomposed efficiently. For example, UV-B having the wavelength of 280 nm or more and 315 nm or less and/or UV-C having the wavelength of 180 nm or more and 280 nm or less may be used, the light containing UV-C having the wavelength of 180 nm or more and 280 nm or less is preferably used, and the light of which peak wavelength is included in the ranges is also preferred.

Since the gaseous C₂₋₄ halogenated hydrocarbon is oxidatively photodecomposed in the present invention, even the high energy light having relatively low energy may possibly oxidatively photodecompose the C₂₋₄ halogenated hydrocarbon. In particular, when the C₂₋₄ halogenated hydrocarbon that does not contain a stabilizer is used, even the high energy light having relatively low energy may possibly oxidatively photodecompose the C₂₋₄ halogenated hydrocarbon. An example of the high energy light having relatively low energy includes the light of which peak wavelength is included in visible light wavelength range. The visible light wavelength range may be 350 nm or more and 830 nm or less, and is preferably 360 nm or more, more preferably 380 nm or more, even more preferably 400 nm or more, and preferably 800 nm or less, more preferably 780 nm or less, even more preferably 500 nm or less.

A means for the light irradiation is not particularly restricted as long as the light having the above-described wavelength can be irradiated by the means. An example of a light source of the light having such a wavelength range includes sunlight, low pressure mercury lamp, medium pressure mercury lamp, high pressure mercury lamp, ultrahigh pressure mercury lamp, chemical lamp, black light lamp, metal halide lamp and LED lamp. A low pressure mercury lamp is preferably used in terms of a reaction efficiency and a cost.

The conditions such as a strength of the irradiation light may be appropriately determined depending on the C₂₋₄ halogenated hydrocarbon or the like. For example, a light strength at the shortest distance position of the flowed mixed gas from the light source is determined depending on the production scale and the wavelength of the irradiation light and is preferably 1 mW/cm² or more and 200 mW/cm² or less. For example, when the wavelength of the irradiation light is relatively short, the light strength is more preferably 100 mW/cm² or less or 50 mW/cm² or less and even more preferably 20 mW/cm² or less or 10 mW/cm² or less. When the wavelength of the irradiation light is relatively long, the light strength is more preferably 10 mW/cm² or more and 20 mW/cm² or more and may be 50 mW/cm² or more or 100 mW/cm² or more. The shortest distance between the light source and the flowed mixed gas is preferably 1 m or less, more preferably 50 cm or less, and even more preferably 10 cm or less or 5 cm or less. The lower limit of the shortest distance is not particularly restricted and may be 0 cm. In other words, the light source is placed in the flowed mixed gas.

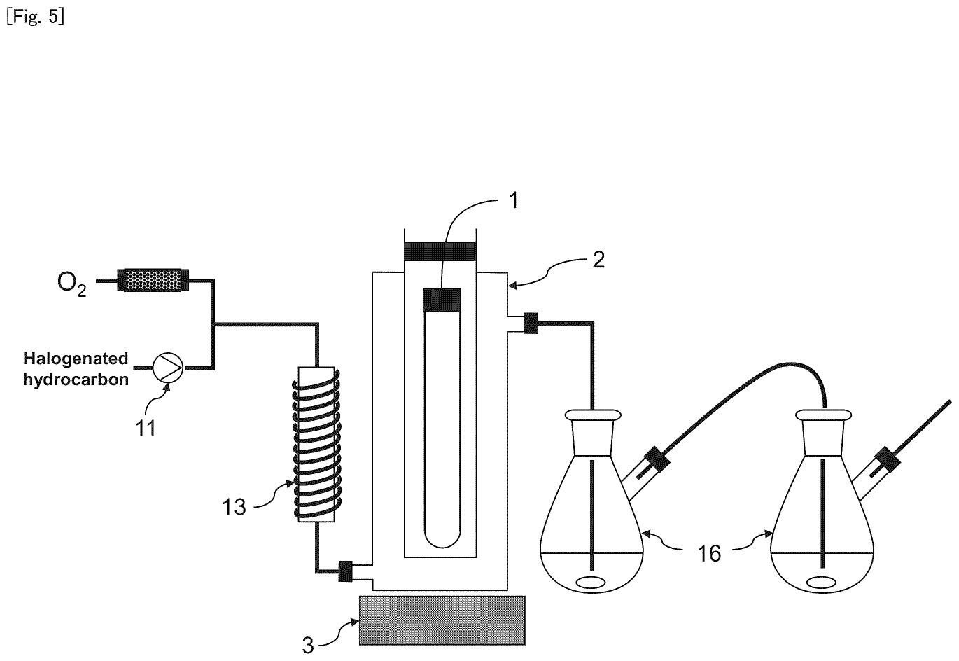

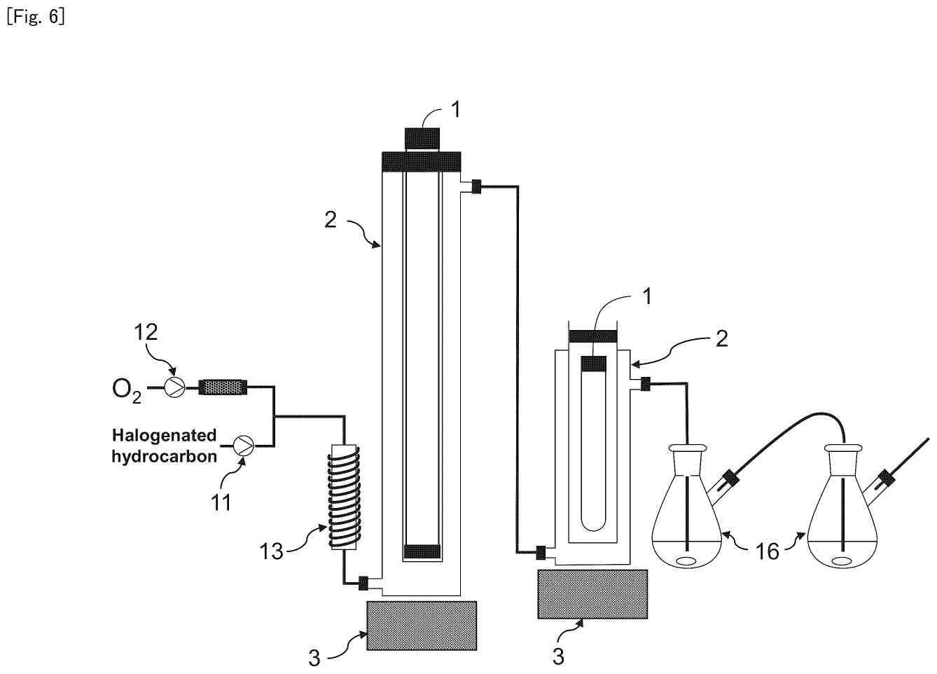

The conditions to irradiate the high energy light to the flowed mixed gas are not particularly restricted. For example, the photoreaction vessel 2 having the light source 1 inside is constructed, the C₂₋₄ halogenated hydrocarbon is vaporized in the photoreaction vessel 2, and high energy light may be irradiated from the light source 1 with supplying an oxygen-containing gas into the photoreaction vessel 2 as demonstrated in FIG. 1. Also, the vaporized C₂₋₄ halogenated hydrocarbon and an oxygen-containing gas may be supplied into the photoreaction vessel 2 as demonstrated in FIGS. 5 and 6. Alternatively, the flow photoreaction device 14 is constructed by arranging one or more reaction tubes around the light source, or the flow photoreaction device 14 may has a gas inlet and a gas outlet at both ends and the light source is inserted thereinto. The mixed gas may be supplied through the flow photoreaction device 14 as demonstrated in FIGS. 2 to 4. A reaction tube may be spirally wrapped around the light source in order to efficiently irradiate high energy light to the mixed gas in the flow photoreaction device 14. The flow photoreaction device 14 may be equipped with a heating means to maintain the gas state of the C₂₋₄ halogenated hydrocarbon. An example of such a heating means includes a hot bath into which a part or all of the flow photoreaction device 14 can be immersed and a heater that can heat a part or all of the outside of the flow photoreaction device 14.

The vaporized C₂₋₄ halogenated hydrocarbon may be oxidatively photodecomposed to a carbonyl halide by oxygen and the high energy light. It also has been known that a carbonyl halide is decomposed by high energy light. Thus, it is important to adjust the conditions to irradiate the high energy light in order not to excessively decompose the produced carbonyl halide.

For example, the time to irradiate the above-described high energy light to the above-described flowed mixed gas may be adjusted depending on the wavelength of the irradiation light and the reaction temperature, and is preferably 1 second or more and 2000 seconds or less. The time to irradiate the high energy light corresponds to the retention time of the flowed mixed gas in the photoreaction vessel in which the high energy light is continuously irradiated to the flowed mixed gas. When the time is 1 second or more, the vapored C₂₋₄ halogenated hydrocarbon can be oxidatively photodecomposed more surely. When the time is 2000 seconds or less, the excessive decomposition of the produced carbonyl halide may be inhibited more surely. The time is preferably 5 seconds or more, more preferably 10 seconds or more, even more preferably 20 seconds or more or 30 seconds or more, and preferably 1500 seconds or less, 1000 seconds or less, 500 seconds or less or 300 seconds or less, more preferably 100 seconds or less, even more preferably 60 seconds or less or 50 seconds or less. When the C₂₋₄ halogenated hydrocarbon that does not contain a stabilizer is used, the decomposition of the produced carbonyl halide can be further inhibited by using the light of which wavelength is relatively long. The light irradiation time may be adjusted within 1 second or more and 10000 seconds or less in such a case. The light irradiation time is preferably 5000 seconds or less and more preferably 1000 seconds or less in terms of the production efficiency.

The time to irradiate the high energy light is longer, the C₂₋₄ halogenated hydrocarbon may be decomposed more efficiently but the produced carbonyl halide may be further oxidatively photodecomposed. On the one hand, when the oxygen concentration in the mixed gas is adjusted to be low, the C₂₋₄ halogenated hydrocarbon may be oxidatively photodecomposed with suppressing the further oxidative photodecomposition of the carbonyl halide. For example, when the oxygen concentration in the mixed gas is adjusted to 15±5 vol %, preferably 15±2 vol %, the time to irradiate light to the mixed gas may be adjusted to 50 seconds or more, 100 seconds or more, 150 seconds or more, 200 seconds or more, 500 seconds or more or 1000 seconds or more.

The flow rate of the flowed mixed gas in the photoreaction vessel to irradiate high energy light to the flowed mixed gas is preferably determined in terms of the internal volume of the photoreaction vessel. For example, when the internal volume of the photoreaction vessel is large, the residence time of the mixed gas tends to become longer and thus the flow rate is preferably adjusted to be increased. On the one hand, when the internal volume of the photoreaction vessel is small, the flow rate is preferably adjusted to be reduced. Specifically, since the internal volume of the photoreaction vessel (L)/the flow rate of the flowed mixed gas (L/s) corresponds to the residence time of the flowed mixed gas (s), the flow rate of the flowed mixed gas can be determined in terms of the desired residence time and the internal volume of the photoreaction vessel. The flow rate of the flowed mixed gas can be regarded as the same as the flow rate of the oxygen-containing gas in the embodiment demonstrated in FIG. 1.

The linear velocity of the flowed mixed gas in the photoreaction vessel may be adjusted to 0.001 m/min or more and 100 m/min or less. When the linear velocity is 0.001 m/min or more, the photodecomposition of the carbonyl halide produced from the C₂₋₄ halogenated hydrocarbon by the gas phase reaction can be inhibited more surely. When the linear velocity is 100 m/min or less, the time to transform the C₂₋₄ halogenated hydrocarbon to a carbonyl halide can be sufficiently obtained more surely. The linear velocity can be calculated by dividing the flow rate of the flowed mixed gas passing through the photoreaction vessel by the cross-sectional area of the photoreaction vessel. When the cross-sectional area of the photoreaction vessel is not constant, the cross-sectional area may be regarded as the average value of the cross-sectional areas of the photoreaction vessel in the moving direction of the flowed mixed gas. The average value can be calculated by dividing the internal volume of the photoreaction vessel by the length of the photoreaction vessel in the moving direction of the flowed mixed gas. The linear velocity is preferably 0.01 m/min or more, more preferably 0.1 m/min or more, and preferably 50 m/min or less or 20 m/min or less, more preferably 10 m/min or less or 5 m/min or less, even more preferably 1 m/min or less or 0.5 m/min or less.

The temperature during the irradiation of the high energy light to the vaporized C₂₋₄ halogenated hydrocarbon may be appropriately adjusted as long as the vaporization of the C₂₋₄ halogenated hydrocarbon can be maintained and the excessive decomposition of the produced carbonyl halide can be inhibited, and may be adjusted to, for example, 100° C. or higher and 300° C. or lower. The temperature is preferably 110° C. or higher, more preferably 120° C. or higher, and preferably 250° C. or lower, more preferably 200° C. or lower. The temperature may be adjusted by adjusting the temperature of the vaporized C₂₋₄ halogenated hydrocarbon and/or the temperature of the oxygen-containing gas supplied into the reaction vessel. The reaction vessel may be heated using a heat medium to maintain the temperature of the mixed gas in the reaction vessel.

When the high energy light is irradiated to the C₂₋₄ halogenated hydrocarbon, the mixed gas containing the C₂₋₄ halogenated hydrocarbon and oxygen may not be pressurized but may be pressurized to the extent that at least the mixed gas can pass through the reaction vessel. In addition, the productivity may be improved by pressurizing the mixed gas. The gauge pressure of the mixed gas in the reaction vessel may be adjusted to 0 MPaG or more and 2 MPaG or less, and is preferably 1 MPaG or less and more preferably 0.5 MPaG or less.

The C₂₋₄ halogenated hydrocarbon is oxidatively photodecomposed and a carbonyl halide [X—C(═O)—X wherein X is one or more halogeno groups selected from the group consisting of chloro, bromo and iodo.] may be produced. A carbonyl halide-like compound which plays a similar role to a carbonyl halide may be also produced in addition to a carbonyl halide. The carbonyl halide-like compound is included in the carbonyl halide of the present invention. The representative reactions in which the carbonyl halide is used is hereinafter described.

3. Post Reaction Step—Production of Carbonate Compound

A carbonate compound can be produced by reacting the carbonyl halide and an alcohol compound.

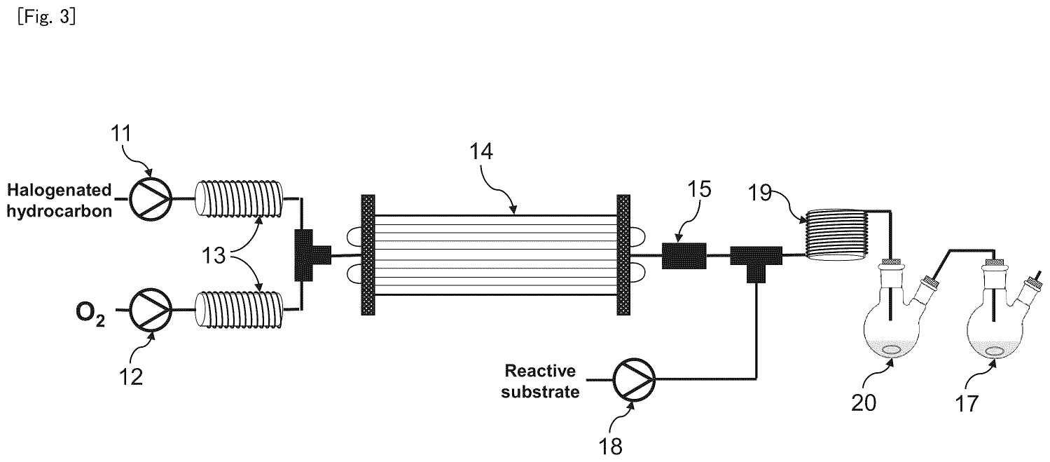

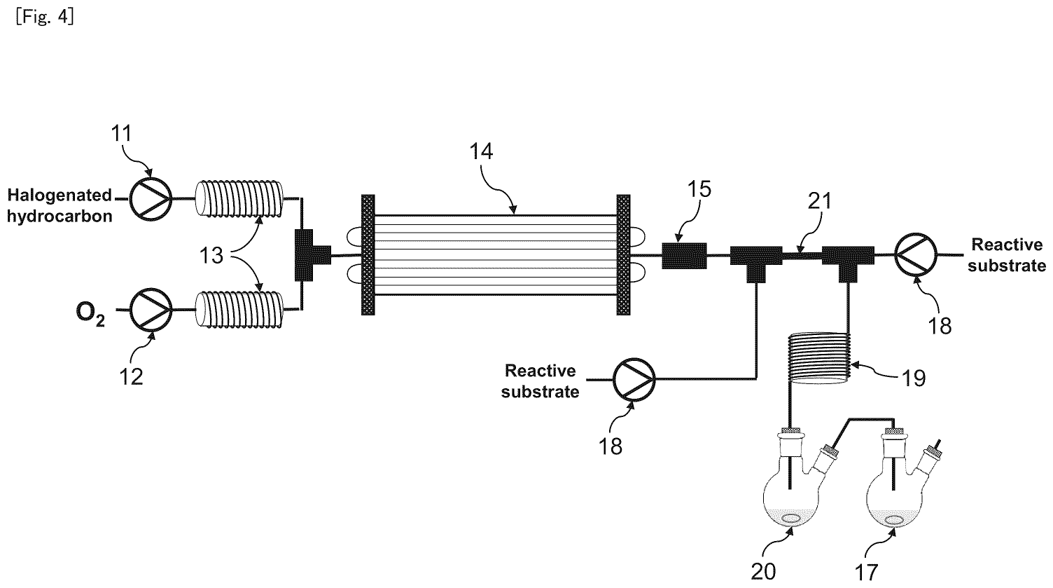

The conditions of the reaction are not particularly restricted. For example, the carbonyl halide produced in the photoreaction vessel 2 is pushed out from the photoreaction vessel 2 by supplying the oxygen-containing gas and blown into the composition containing an alcohol compound in the reaction vessel 6 through the cooling condenser 5-1 as demonstrated in FIG. 1. The temperature of the cooling condenser is preferably adjusted so that the produced carbonyl halide can pass therethrough in this case. For example, since the boiling point of phosgene among a carbonyl halide is 8.2° C., the temperature of the cooling condenser 5-1 is preferably adjusted to 10° C. or more in the case of phosgene. The gas containing the carbonyl halide may be blown into the composition containing an alcohol compound without passing through a cooling condenser as demonstrated in FIGS. 5 and 6. Alternatively, the gas containing the produced carbonyl halide may be blown into the composition containing an alcohol compound in the reaction vessel 16 as demonstrated in FIG. 2. The flow photoreaction device 14 is preferably heated to inhibit the devolatilization of the C₂₋₄ halogenated hydrocarbon in this case. Furthermore, an alcohol compound may be supplied into the temperature-adjustable coil reaction device 19 to react the carbonyl halide and the alcohol compound in the coil reaction device as demonstrated in FIG. 3 and FIG. 4. An alcohol compound may be vaporized by adjusting the temperature of the coil reaction device in order to react the carbonyl halide and the alcohol compound in a gas phase in this case.

An alcohol compound means an organic compound having a hydroxy group and may be exemplified by a monovalent alcohol compound represented by the following formula (I) or a divalent alcohol compound represented by the following formula (II). Hereinafter, the compound represented by formula x is abbreviated as “compound x” in some cases. For example, a monovalent alcohol compound represented by the formula (I) is abbreviated as the “monovalent alcohol compound (I)” in some cases.

R¹—OH (I)

HO—R²—OH (II)

wherein R¹ is a monovalent organic group and R² is a divalent organic group.

An organic group is not particularly restricted as long as the organic group is inactive in the reaction of this step and is exemplified by an optionally substituted C₁₋₁₀ aliphatic hydrocarbon group, an optionally substituted C₆₋₁₂ aromatic hydrocarbon group, an optionally substituted heteroaryl group, an organic group constructed by binding 2 or more and 5 or less of an optionally substituted C₁₋₁₀ aliphatic hydrocarbon group and an optionally substituted C₆₋₁₂ aromatic hydrocarbon group, and an organic group constructed by binding 2 or more and 5 or less of an optionally substituted C₁₋₁₀ aliphatic hydrocarbon group and an optionally substituted heteroaryl group.

An example of the C₁₋₁₀ aliphatic hydrocarbon group includes a C₁₋₁₀ chain aliphatic hydrocarbon group, a C₃₋₁₀ cyclic aliphatic hydrocarbon group, and an organic group constructed by binding 2 or more and 5 or less of a C₁₋₁₀ chain aliphatic hydrocarbon group and a C₃₋₁₀ cyclic aliphatic hydrocarbon group.

The “C₁₋₁₀ chain aliphatic hydrocarbon group” means a linear or branched saturated or unsaturated aliphatic hydrocarbon group having the carbon number of 1 or more and 10 or less. An example of the monovalent C₁₋₁₀ chain aliphatic hydrocarbon group includes a C₁₋₁₀ alkyl group, a C₂₋₁₀ alkenyl group and a C₂₋₁₀ alkynyl group.

An example of the C₁₋₁₀ alkyl group includes methyl, ethyl, n-propyl, isopropyl, n-butyl, 1-methylpropyl, 2-methylpropyl, 1,1-dimethylethyl, 2,2-dimethylethyl, n-pentyl, n-hexyl, 2-hexyl, 3-hexyl, 4-methyl-2-pentyl, n-heptyl, n-octyl and n-decyl. The C₁₋₁₀ alkyl group is preferably a C₂₋₈ alkyl group and more preferably a C₄₋₆ alkyl group.

An example of the C₂₋₁₀ alkenyl group includes ethenyl (vinyl), 1-propenyl, 2-propenyl (allyl), butenyl, hexenyl, octenyl and decenyl. The C₂₋₁₀ alkenyl group is preferably a C₂-₈ alkenyl group and more preferably a C₄₋₆ alkenyl group.

An example of the C₂₋₁₀ alkynyl group includes ethinyl, propynyl, butynyl, hexynyl, octynyl and pentadecynyl. The C₂₋₁₀ alkynyl group is preferably a C₂₋₈ alkynyl group and more preferably a C₂₋₆ alkynyl group.

The “C₃₋₁₀ cyclic aliphatic hydrocarbon group” means a cyclic saturated or unsaturated aliphatic hydrocarbon group having the carbon number of 3 or more and 10 or less. An example of the monovalent C₃₋₁₀ cyclic aliphatic hydrocarbon group includes a C₃₋₁₀ cycloalkyl group, a C₄₋₁₀ cycloalkenyl group and a C₄₋₁₀ cycloalkynyl group.

An example of the organic group constructed by binding 2 or more and 5 or less of the C₁₋₁₀ chain aliphatic hydrocarbon group and the C₃₋₁₀ cyclic aliphatic hydrocarbon group includes a monovalent C₃₋₁₀ cyclic aliphatic hydrocarbon group—divalent C₁₋₁₀ chain aliphatic hydrocarbon group, and a monovalent C₁₋₁₀ chain aliphatic hydrocarbon group—divalent C₃₋₁₀ cyclic aliphatic hydrocarbon group—divalent C₁₋₁₀ chain aliphatic hydrocarbon group.

The “C₆₋₁₂ aromatic hydrocarbon group” means an aromatic hydrocarbon group having the carbon number of 6 or more and 12 or less. An example of the monovalent C₆₋₁₂ aromatic hydrocarbon group includes phenyl, indenyl, naphthyl and biphenyl, and phenyl is preferred.

The “heteroaryl group” means a 5-membered aromatic heterocyclic group, a 6-membered aromatic heterocyclic group and a condensed ring aromatic heterocyclic group having at least one hetero atom such as a nitrogen atom, an oxygen atom and a sulfur atom. An example of the heteroaryl group includes a monovalent 5-membered aromatic heterocyclic group such as pyrrolyl, imidazolyl, pyrazolyl, thienyl, furyl, oxazolyl, isoxazolyl, thiazolyl, isothiazolyl and thiadiazolyl; a monovalent 6-membered aromatic heterocyclic group such as pyridinyl, pyrazinyl, pyrimidinyl and pyridazinyl; and a condensed ring aromatic heterocyclic group such as indolyl, isoindolyl, quinolinyl, isoquinolinyl, benzofuranyl, isobenzofuranyl and chromenyl.

An example of the “organic group constructed by binding 2 or more and 5 or less of the C₁₋₁₀ aliphatic hydrocarbon group and the C₆₋₁₂ aromatic hydrocarbon group” includes a C₆₋₁₂ aromatic hydrocarbon group—C₁₋₁₀ chain aliphatic hydrocarbon group, a C₁₋₁₀ chain aliphatic hydrocarbon group—C₆₋₁₂ aromatic hydrocarbon group, a C₁₋₁₀ chain aliphatic hydrocarbon group —C₆₋₁₂ aromatic hydrocarbon group—C₁₋₁₀ chain aliphatic hydrocarbon group, and a C₆₋₁₂ aromatic hydrocarbon group—C₁₋₁₀ chain aliphatic hydrocarbon group—C₆₋₁₂ aromatic hydrocarbon group. An example of the “organic group constructed by binding 2 or more and 5 or less of the C₁₋₁₀ aliphatic hydrocarbon group and the heteroaryl group” includes a heteroaryl group—C₁₋₁₀ chain aliphatic hydrocarbon group, a C₁₋₁₀ chain aliphatic hydrocarbon group—heteroaryl group, a C₁₋₁₀ chain aliphatic hydrocarbon group—heteroaryl group—C₁₋₁₀ chain aliphatic hydrocarbon group, and a heteroaryl group—C₁₋₁₀ chain aliphatic hydrocarbon group—heteroaryl group.

An example of the substituent group that the C₁₋₁₀ aliphatic hydrocarbon group may optionally have includes one or more substituent groups selected from the group consisting of a halogeno group, a nitro group and a cyano group, and a halogeno group is preferred. An example of the substituent group that the C₆₋₁₂ aromatic hydrocarbon group and the heteroaryl group may optionally have includes one or more substituent groups selected from the group consisting of a C₁₋₆ alkyl group, a C₁₋₆ alkoxy group, a halogeno group, a nitro group and a cyano group, and a halogeno group is preferred. An example of the halogeno group includes fluoro, chloro, bromo and iodo, and fluoro is preferred.

An alcohol compound may be classified into a fluorinated alcohol compound and a non-fluorinated alcohol compound. The fluorinated alcohol compound indispensably has a fluoro group as a substituent group, and the non-fluorinated alcohol compound is not substituted with a fluoro group. The halogeno group that a non-fluorinated alcohol compound may optionally have is one or more halogeno groups selected from chloro, bromo and iodo. The group “R^{x}” having a fluoro as substituent group may be described as “R_{F}^{x}”.

The “C₁₋₆ alkyl group” means a linear or branched monovalent saturated aliphatic hydrocarbon group having the carbon number of 1 or more and 6 or less. An example of the C₁₋₆ alkyl group includes methyl, ethyl, n-propyl, isopropyl, n-butyl, isobutyl, s-butyl, t-butyl, n-pentyl and n-hexyl. The C₁₋₆ alkyl group is preferably a C₁₋₄ alkyl group, more preferably a C₁₋₂ alkyl group and even more preferably methyl.

The “C₁₋₆ alkoxy group” means a linear or branched saturated aliphatic hydrocarbon oxy group having the carbon number of 1 or more and 6 or less. An example of the C₁₋₆ alkoxy group includes methoxy, ethoxy, n-propoxy, isopropoxy, n-butoxy, isobutoxy, t-butoxy, n-pentoxy and n-hexoxy. The C₁₋₆ alkoxy group is preferably a C₁₋₄ alkoxy group, more preferably a C₁₋₂ alkoxy group and even more preferably methoxy.

The monovalent alcohol compound (I) may be a fluorinated alcohol compound. An example of the monovalent fluorinated alcohol compound (I) includes a fluorinated ethanol such as difluoroethanol and trifluoroethanol; a fluorinated propanol such as monofluoropropanol, difluoropropanol, trifluoropropanol, tetrafluoropropanol, pentafluoropropanol and hexafluoropropanol.

An example of the divalent organic group includes divalent organic groups derived from the examples of the monovalent organic group. For example, the divalent organic group derived from the C₁₋₁₀ alkyl group, the C₂₋₁₀ alkenyl group and the C₂₋₁₀ alkynyl group as the monovalent organic group is a C₁₋₁₀ alkane-diyl group, a C₂₋₁₀ alkene-diyl group and a C₂₋₁₀ alkyne-diyl group.

The divalent organic group may be a divalent (poly)alkylene glycol group: —[—O—R²—]ⁿ— wherein R² is a C₁₋₈ alkane-diyl group, and n is an integer of 1 or more and 50 or less.

In addition, an example of the divalent alcohol compound (II) includes the following divalent alcohol compound (II-1):

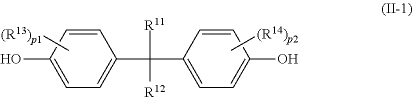

wherein
R¹¹ and R¹² are independently H, a C₁₋₆ alkyl group, a C₁₋₆ fluoroalkyl group or a C₆₋₁₂ aromatic hydrocarbon group, or form a C₃₋₆ cycloalkyl group optionally having a C₁₋₆ alkyl as a substituent together with each other,
R¹³ and R¹⁴ are independently H, a C₁₋₆ alkyl group or a C₆₋₁₂ aromatic hydrocarbon group, and when p1 or p2 is an integer of 2 or more, a plurality of R¹³ or R¹⁴ are the same as or different from each other,
p1 and p2 are independently integers of 0 or more and 4 or less.

An example of the specific divalent non-fluorinated alcohol compound (II-1) includes 2,2-bis(4-hydroxyphenyl)propane, 1,1-bis(4-hydroxyphenyl)-1-phenylethane, 2,2-bis(4-hydroxyphenyl)hexafluoropropane, 2,2-bis(4-hydroxyphenyl)butane, bis(4-hydroxyphenyl)diphenylmethane, 2,2-bis(3-methyl-4-hydroxyphenyl)propane, 1,1-bis(4-hydroxyphenyl)ethane, bis(4-hydroxyphenyl)methane and 2,2-bis(4-hydroxy-3-isopropylphenyl)propane, and 2,2-bis(4-hydroxyphenyl)propane, i.e. Bisphenol A, is preferred.

The divalent alcohol compound (II) may be a fluorinated alcohol compound. An example of such a divalent fluorinated alcohol compound (II) includes fluorinated ethylene glycol; a fluorinated propylene glycol such as monofluoropropylene glycol and difluoropropylene glycol; a fluorinated butanediol such as monofluorobutanediol, difluorobutanediol, trifluorobutanediol and tetrafluorobutanediol; a fluorinated pentanediol such as monofluoropentanediol, difluoropentanediol, trifluoropentanediol, tetrafluoropentanediol, pentafluoropentanediol and hexafluoropentanediol; a fluorinated hexanediol such as monofluorohexanediol, difluorohexanediol, trifluorohexanediol, tetrafluorohexanediol, pentafluorohexanediol, hexafluorohexanediol, heptafluorohexanediol and octafluorohexanediol; a fluorinated heptanediol such as monofluoroheptanediol, difluoroheptanediol, trifluoroheptanediol, tetrafluoroheptanediol, pentafluoroheptanediol, hexafluoroheptanediol, heptafluoroheptanediol, octafluoroheptanediol, nonafluoroheptanediol and decafluoroheptanediol; a fluorinated octanediol such as monofluorooctanediol, difluorooctanediol, trifluorooctanediol, tetrafluorooctanediol, pentafluorooctanediol, hexafluorooctanediol, heptafluorooctanediol, octafluorooctanediol, nonafluorooctanediol, decafluorooctanediol, undecafluorooctanediol and dodecafluorooctanediol; a fluorinated nonanediol such as monofluorononanediol, difluorononanediol, trifluorononanediol, tetrafluorononanediol, pentafluorononanediol, hexafluorononanediol, heptafluorononanediol, octafluorononanediol, nonafluorononanediol, decafluorononanediol, undecafluorononanediol, dodecafluorononanediol, tridecafluorononanediol and tetradecafluorononanediol; a fluorinated decanediol such as monofluorodecanediol, difluorodecanediol, trifluorodecanediol, tetrafluorodecanediol, pentafluorodecanediol, hexafluorodecanediol, heptafluorodecanediol, octafluorodecanediol, nonafluorodecanediol, decafluorodecanediol, undecafluorodecanediol, dodecafluorodecanediol, tridecafluorodecanediol, tetradecafluorodecanediol, pentadecafluorodecanediol and hexadecafluorodecanediol; a fluorinated polyethylene glycol such as fluorinated diethylene glycol, fluorinated triethylene glycol, fluorinated tetraethylene glycol, fluorinated pentaethylene glycol and fluorinated hexaethylene glycol.

The usage amount of the alcohol compound may be appropriately adjusted as long as the reaction successfully proceeds. For example, 1 or more molar ratio of the divalent alcohol compound to the produced carbonyl halide may be used, and 2 or more molar ratio of the monovalent alcohol compound may be used to the produced carbonyl halide. A carbonate compound can be produced more efficiently by using excessive amount of the alcohol compound. Since the yield of the carbonyl halide to the used C₂₋₄ halogenated hydrocarbon is not constant, the molar ratio of the divalent alcohol compound to the C₂₋₄ halogenated hydrocarbon is preferably adjusted to 1 or more and the molar ratio of the monovalent alcohol compound to the C₂₋₄ halogenated hydrocarbon is preferably adjusted to 2 or more. The molar ratio of the divalent alcohol is preferably 1.5 or more, more preferably 2 or more, and preferably 10 or less, more preferably 5 or less. The molar ratio of the monovalent alcohol is preferably 2 or more, more preferably 4 or more, and preferably 20 or less, more preferably 10 or less.

A base may be used for accelerating the reaction of the carbonyl halide and the alcohol compound. A base is classified into an inorganic base and an organic base. An example of the inorganic base includes a carbonate salt of an alkali metal, such as lithium carbonate, sodium carbonate, potassium carbonate and cesium carbonate; a carbonate salt of a Group 2 metal, such as magnesium carbonate, calcium carbonate and barium carbonate; a hydrogencarbonate salt of an alkali metal, such as lithium hydrogencarbonate, sodium hydrogencarbonate, potassium hydrogencarbonate and cesium hydrogencarbonate; a hydroxide of an alkali metal, such as lithium hydroxide, sodium hydroxide and potassium hydroxide; a hydroxide of a Group 2 metal, such as magnesium hydroxide and calcium hydroxide; a fluoride salt of an alkali metal, such as lithium fluoride, sodium fluoride, potassium fluoride and cesium fluoride. A carbonate salt or a hydrogencarbonate salt of an alkali metal or a Group 2 metal is preferred due to low moisture absorbency and low deliquescency, and a carbonate salt of an alkali metal is more preferred. For example, a tri(C₁₋₄ alkyl)amine such as trimethylamine, triethylamine and diisopropylethylamine; a tert-butoxide of an alkali metal, such as sodium tert-butoxide and potassium tert-butoxide; a non-nucleophilic organic base such as diazabicycloundecene, lithium diisopropylamide, lithium tetramethylpiperidine, 1,4-diazabicyclo[2.2.2]octane (DABCO), 1,5,7-triazabicyclo[4.4.0]dec-5-ene (TBD), 7-methyl-1,5,7-triazabicyclo[4.4.0]dec-5-ene (MTBD), 1,8-diazabicyclo[5.4.0]undec-7-ene (DBU), 1,5-diazabicyclo[4.3.0]non-5-ene (DBN), 1,1,3,3-tetramethylguanidine (TMG) and N-methylmorpholine can be used as an organic base in terms of low reactivity with the product by the photoreaction of tetrahaloethylene. Also, low-nucleophilic organic base such as pyridine and lutidine may be used.

A hydrogen halide such as hydrogen chloride is produced as a side product during an oxidative photodecomposition reaction of a C₂₋₄ halogenated hydrocarbon and the reaction of a carbonyl halide and an alcohol compound. A base is useful for capturing such a hydrogen halide. But when a reaction tube having a small diameter, such as a coil reaction device demonstrated in FIG. 3 and FIG. 4, is used, a salt of a hydrogen halide and a base may precipitate and thus the reaction tube may become clogged in some cases. The base of which salt with a hydrogen halide is an ionic liquid is preferably used in such a case. An example of the base includes an organic base such as an imidazole derivative, for example, 1-methylimidazole. In addition, the base of which hydrochloride salt has a relatively low melting point, such as pyridine, may be used.

The usage amount of the base may be appropriately adjusted as long as the reaction successfully proceeds, and for example, the usage amount to 1 mol of the C₂₋₄ halogenated hydrocarbon may be adjusted to 1 mol or more and 10 mol or less.

For example, the base may be added to the alcohol compound, or the base may be continuously supplied with the alcohol compound.

When the carbonyl halide and the alcohol compound are reacted, a solvent may be used. For example, a solvent may be added to the composition containing the alcohol compound. An example of the solvent includes a ketone solvent such as acetone, methyl ethyl ketone, methyl isobutyl ketone and cyclohexanone; an ester solvent such as ethyl acetate; an aliphatic hydrocarbon solvent such as n-hexane; an aromatic hydrocarbon solvent such as benzene, toluene, xylene and benzonitrile; an ether solvent such as diethyl ether, tetrahydrofuran and dioxane; a nitrile solvent such as acetonitrile; and a halogenated hydrocarbon solvent such as dichloromethane and chloroform.

The temperature for reacting the carbonyl halide and the alcohol compound is not particularly restricted and may be appropriately adjusted. For example, the temperature may be adjusted to 0° C. or higher and 120° C. or lower. The temperature is more preferably 10° C. or higher, even more preferably 20° C. or higher, and more preferably 100° C. or lower, even more preferably 80° C. or lower or 50° C. or lower. When the base is not used or the base is used to further accelerate the reaction, the temperature may be adjusted to be relatively higher, for example, 50° C. or higher or 100° C. or higher.

The time to react the carbonyl halide and the alcohol compound is not particularly restricted and may be appropriately adjusted. For example, the time is preferably 0.5 hours or more and 50 hours or less. The reaction time is more preferably 1 hour or more, even more preferably 5 hours or more, and more preferably 30 hours or less, even more preferably 20 hours or less. For example, even after the production of the carbonyl halide is finished, the reaction mixture may be continuously stirred until the consumption of the alcohol compound is confirmed.

When the monovalent alcohol compound (I) is used, the chain carbonate compound represented by the following formula (III) is produced by the reaction of the carbonyl halide and the alcohol compound. When the divalent alcohol compound (II) is used, the polycarbonate compound comprising the unit represented by the following formula (IV-1) or the cyclic carbonate compound represented by the following formula (IV-2) is produced. When the divalent alcohol compound (II) is used, whether the polycarbonate compound (IV-1) is produced or the cyclic carbonate compound (IV-2) is produced and the production ratio thereof are mainly dependent on the distance between two hydroxy groups and the flexibility of the chemical structure of the divalent alcohol compound (II) and may be specifically determined by a preliminary experiment or the like.

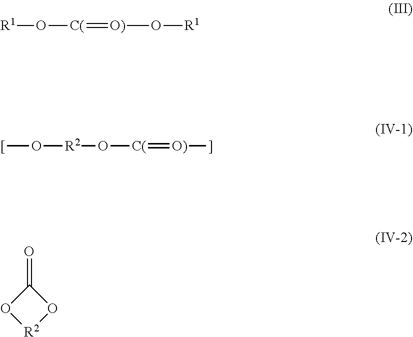

4. Post-Reaction Step—Production of Halogenated Formic Acid Ester

A halogenated formic acid ester can be produced by adjusting the molar ratio of the alcohol compound to the C₂₋₄ halogenated hydrocarbon to less than 1 without using a base in the above-described method for producing a carbonate compound. The molar ratio is preferably 0.9 or less and more preferably 0.8 or less.

5. Post-Reaction Step—Production of Isocyanate Compound

An isocyanate compound can be produced by reacting the carbonyl halide and a primary amine compound. An isocyanate compound is useful as a raw material of a carbamate compound, a urethane compound or the like. A primary amine compound may be used in place of the alcohol compound in the above-described method for producing a carbonate compound except for the following points as the reaction embodiment.

A primary amine compound is not particularly restricted as long as the compound has 1 or more amino groups (—NH₂ groups). An example of the primary amine compound includes the primary amine compound (V): R³—(NH₂)ₘ wherein R³ is a m-valent organic group, and m is integer of 1 or more and 6 or less, preferably 5 or less, 4 or less or 3 or less, more preferably 1 or 2, and even more preferably 2.

An example of a monovalent organic group among the organic group R³ includes the same group as the monovalent organic group R¹ in the above-described method for producing a carbonate compound. An example of a divalent organic group includes the same group as the divalent organic group R². An example of a tri or more-valent organic group includes a tri or more-valent organic group derived from the examples of the monovalent organic group R¹. For example, a trivalent organic group derived from a C₁₋₁₀ alkyl group, a C₂₋₁₀ alkeny group and a C₂₋₁₀ alkynyl group as a monovalent organic group is a C₁₋₁₀ alkane triyl group, a C₂₋₁₀ alkene triyl group and a C₂₋₁₀ alkyne triyl group.

The isocyanate compound (VI): R³—(N═C═O)ₘ can be produced by reacting the carbonyl halide and the primary amine compound (V). The produced R³—(N═C═O)ₘ may be possibly reacted with the primary amine compound (V) to produce a urea compound, such as R³—[NH—C(═O)—NH—R³—(NH₂)ₘ₋₁]ₘ, and it is preferred to inhibit the reaction that the molar ratio of the primary amine compound (V) to the C₂₋₄ halogenated hydrocarbon is adjusted to 1 or less, a salt is used as the primary amine compound (V), or a base is not used. In addition, an isocyanate compound can be efficiently produced by the following conditions: the produced carbonyl halide is dissolved in a solvent to prepare a carbonyl halide solution, and the molar ratio of the carbonyl halide to the primary amine compound (V) is maintained at more than 1 by adding the primary amine compound (V) or a solution thereof to the carbonyl halide solution.

When the target compound is an isocyanate compound, the molar ratio of the primary amine compound (V) to the produced carbonyl halide is preferably adjusted to 1 or less. Since it may be difficult in some cases to predict the accurate amount of the produced carbonyl halide, the molar ratio of the primary amine compound (V) to the used C₂₋₄ halogenated hydrocarbon is preferably adjusted to less than 1. The molar ratio is preferably 0.5 or less, more preferably 0.2 or less, and preferably 0.001 or more, more preferably 0.05 or more. When the target compound is a urea compound, the ratio is preferably 2 or more, more preferably 4 or more, and preferably 20 or less, more preferably 15 or less.

When the target compound is an isocyanate compound, a salt as the primary amine compound (V) is preferably used, since an isocyanate compound is hardly reacted with an amine salt. An example of such a salt includes an inorganic acid salt such as hydrochloride salt, hydrobromide salt, hydroiodide salt, sulfate salt, nitrate salt, perchlorate salt and phosphate salt; and an organic salt such as oxalate salt, malonate salt, maleate salt, fumarate salt, lactate salt, malate salt, citrate salt, tartrate salt, benzoate salt, trifluoroacetate salt, acetate salt, methanesulfonate salt, p-toluenesulfonate salt and trifluoromethanesulfonate salt.

The temperature for the reaction of the carbonyl halide and the primary amine compound is preferably adjusted to be lower than the temperature for the reaction with the alcohol compound in order to maintain the liquid state of the carbonyl halide. For example, the temperature may be adjusted to 15° C. or lower, and is preferably 10° C. or lower, more preferably 5° C. or lower and even more preferably 2° C. or lower. The lower limit of the temperature is not particularly restricted, and the temperature is preferably −80° C. or higher and more preferably −20° C. or higher or −15° C. or higher.

When the target compound is an isocyanate compound and a base is used, the base is preferably 1 or more bases selected from a heterocyclic aromatic amine and a non-nucleophilic strong base. The heterocyclic aromatic amine means a compound that contains at least one hetero ring and at least one amine functional group other than —NH₂. An example of the heterocyclic aromatic amine includes pyridine and a derivative thereof, such as pyridine, α-picoline, β-picoline, γ-picoline, 2,3-lutidine, 2,4-lutidine, 2,6-lutidine, 3,5-lutidine, 2-chloropyridine, 3-chloropyridine, 4-chloropyridine, 2,4,6-trimethylpyridine and 4-dimethylaminopyridine.

The “non-nucleophilic organic base” means a base in which the nucleophilicity of the lone electron pair on the nitrogen atom is weak due to a steric hindrance but of which basicity is strong. An example of the non-nucleophilic organic base includes triethylamine, N,N-diisopropylethylamine, tripropylamine, triisopropylamine, tributylamine, tripentylamine, trihexylamine, triheptylamine, trioctylamine, tridecylamine, tridodecylamine, triphenylamine, tribenzylamine, N,N-diisopropylethylamine, 1,5,7-triazabicyclo[4.4.0]dec-5-ene (TBD), 7-methyl-1,5,7-triazabicyclo[4.4.0]dec-5-ene (MTBD), 1,8-diazabicyclo[5.4.0]undec-7-ene (DBU), 1,5-diazabicyclo[4.3.0]non-5-ene (DBN) and 1,1,3,3-tetramethylguanidine (TMG). In addition, a base of which basicity is relatively high may be used. For example, TBD (pK_{BH+}: 25.98), MTBD (pK_{BH+}: 25.44), DBU (pK_{BH+}: 24.33), DBN (pK_{BH+}: 23.89) and TMG (pK_{BH+}: 23.30) can be used as a base of which basicity (pK_{BH}+) in acetonitrile is 20 or more.

In addition, a versatile organic amine such as trimethylamine, dimethylethylamine, diethylmethylamine, N-ethyl-N-methylbutylamine and 1-methylpyrrolidine can be used as the base.

When the target compound is a urea compound, the molar ratio of the primary amine compound to the C₂₋₄ halogenated hydrocarbon or the produced carbonyl halide is preferably adjusted to more than 1. The molar ratio is preferably 1.5 or more and more preferably 2 or more.

6. Post-Reaction Step—Production of NCA

An amino acid-N-carboxylic anhydride (VIII) (NCA) can be produced by using the amino acid compound (VII) in place of the alcohol compound in the above-described method for producing a carbonate compound.

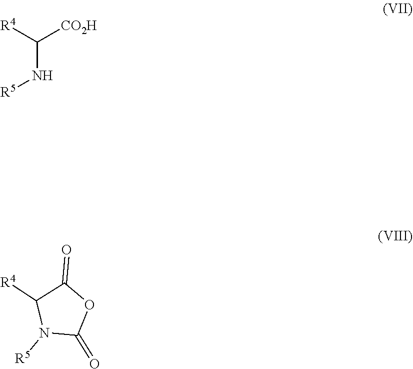

wherein
R⁴ is an amino acid side chain group wherein a reactive group is protected,
R⁵ is H or P¹—[—NH—CHR⁶—C(═O)—]₁— wherein R⁶ is an amino acid side chain group wherein a reactive group is protected, P¹ is a protective group of the amino group, 1 is an integer of 1 or more, and when 1 is an integer of 2 or more, a plurality of R⁶ may be the same as or different from each other.

7. Post-Reaction Step—Production of Vilsmeier Reagent

A Vilsmeier reagent (X) can be produced by reacting the carbonyl halide and the amide compound (IX). A Vilsmeier reagent can be produced similarly to the above-described method for producing a carbonate compound except that the amide compound (IX) is used in place of the alcohol compound and a base is not used.

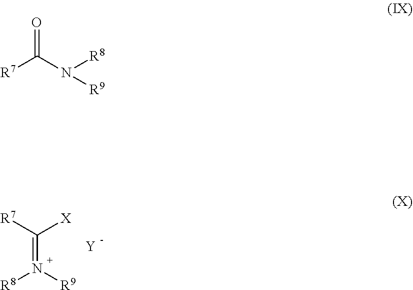

wherein
R⁷ is a hydrogen atom, a C₁₋₆ alkyl group or an optionally substituted C₆₋₁₂ aromatic hydrocarbon group,
R⁸ and R⁹ are independently a C₁₋₆ alkyl group or an optionally substituted C₆₋₁₂ aromatic hydrocarbon group, or R⁸ and R⁹ may form a 4 or more and 7 or less-membered ring structure together with each other,
X is a halogeno group selected from the group consisting of chloro, bromo and iodo,
Y⁻ is a counter anion.

The substituent group that the C₆₋₁₂ aromatic hydrocarbon group may optionally have is not particularly restricted as long as the substituent group does not inhibit the reaction of the present invention, and is exemplified by 1 or more substituent groups selected from the group consisting of a C₁₋₆ alkyl group, a C₁₋₆ alkoxy group, a halogeno group, a nitro group and a cyano group. The number of the substituent group is not particularly restricted as long as the C₆₋₁₂ aromatic hydrocarbon group can be substituted and may be 1 or more and 5 or less. The number is preferably 3 or less, more preferably 2 or less, and even more preferably 1. When the substituent group number is 2 or more, the substituent groups may be the same as or different from each other.

An example of the 4 or more and 7 or less-membered ring structure that is formed by R⁸, R⁹ and the nitrogen atom together with each other includes a pyrrolidyl group, a piperidyl group and a morpholino group.

An example of the specific amide compound (IX) includes N,N-dimethylformamide (DMF), N,N-dimethylacetamide (DMA), N-methyl-N-phenylformamide, N-methylpyrrolidone (NMP), 1,3-dimethylimidazolidinone (DMI), tetramethylurea, tetraethylurea and tetrabutylurea, and DMF is preferred in terms of versatility and cost.

The Y⁻ in the formula (X) is not particularly restricted and is exemplified by a chloride ion, a bromide ion and an iodide ion derived from the C₂₋₄ halogenated hydrocarbon.

The usage amount of the amide compound may be appropriately adjusted as long as the reaction successfully proceeds, and the usage amount to 1 mL of the C₂₋₄ halogenated hydrocarbon may be adjusted to 0.1 mol or more and 100 mol or less.

When the carbonyl halide and the amide compound are reacted, a solvent may be used. For example, a solvent is mixed in a composition containing the amide compound. An example of the solvent includes a ketone solvent such as acetone, methyl ethyl ketone, methyl isobutyl ketone and cyclohexanone; an ester solvent such as ethyl acetate; an aliphatic hydrocarbon solvent such as n-hexane; an aromatic hydrocarbon solvent such as benzene, toluene, xylene and benzonitrile; an ether solvent such as diethyl ether, tetrahydrofuran and dioxane; and a nitrile solvent such as acetonitrile.

The temperature for reacting the carbonyl halide and the amide compound is not particularly restricted and may be appropriately adjusted. For example, the temperature may be adjusted to 0° C. or higher and 120° C. or lower. The temperature is more preferably 10° C. or higher, even more preferably 20° C. or higher, and more preferably 100° C. or lower, even more preferably 80° C. or lower or 50° C. or lower.

The time to react the carbonyl halide and the amide compound is not particularly restricted and may be appropriately adjusted. For example, the time is preferably 0.5 hours or more and 50 hours or less. The reaction time is more preferably 1 hour or more, even more preferably 5 hours or more, and more preferably 30 hours or less, even more preferably 20 hours or less. For example, even after the production of the carbonyl halide is finished, the reaction mixture may be continuously stirred until the consumption of the amide compound is confirmed.

The Vilsmeier-Haack reaction using a Vilsmeier reagent allows the formylation or ketonization of an aromatic compound having an active group. In addition, it has been known that the carboxy group of a carboxylic acid compound can be transformed to a haloformyl group by a Vilsmeier reagent. Furthermore, a formic acid ester can be produced by reacting a Vilsmeier reagent and a hydroxy group-containing compound.

An aromatic compound having an active group means an aromatic compound activated by a substituent group or the like. The aromatic compound is hereinafter described as an “active aromatic compound”. For example, a hydroxy group and an amino group such as an alkylamino group substituted by an alkyl group strongly activate an aromatic compound. In addition, an alkylcarbonylamino group (—N(C═O)R), an alkylcarbonyloxy group (—O(C═O)R), an ether group (—OR), an alkyl group (—R) (R is an alkyl group and is preferably a C₁₋₆ alkyl group) and an aromatic group activate an aromatic compound. The substituent groups are hereinafter referred to as an activating group. In addition, a compound that is constructed by condensing aromatic rings and of which conjugated system is extended, such as anthracene, is also activated and can be subjected to formylation or ketonization by a Vilsmeier reagent. The pi electron at the activated part may be electrophilically reacted with a Vilsmeier reagent to be subjected to formylation or ketonization.

The active aromatic compound is not particularly restricted as long as the active aromatic compound is activated and can be subjected to formylation or ketonization by a Vilsmeier reagent. An example of the active aromatic compound includes a C₆₋₁₀ aromatic hydrocarbon, such as benzene and naphthalene, that may be substituted with the above-described activating group; a condensed aromatic hydrocarbon, such as phenanthrene and anthracene, that may be substituted with the above-described activating group; a 5-membered heteroaryl, such as pyrrol, imidazole, pyrazole, thiophen, furan, oxazole, isoxazole, thiazole, isothiazole and thiadiazole, that may be substituted with the above-described activating group; a 6-membered heteroaryl, such as pyridine, pyrazine, pyrimidine and pyridazine, that may be substituted with the above-described activating group; a condensed heteroaryl, such as indole, isoindole, quinoline, isoquinoline, benzofuran, isobenzofuran and chromene, that may be substituted with the above-described activating group. Though it has not been reported that unsubstituted furan and thiophen are subjected to formylation or ketonization by conventional Vilsmeier-Haack reaction, the formylation or ketonization at the carbon adjacent to a hetero element is possible by the present invention method.

An activating group-containing aromatic compound, a carboxylic acid compound and a hydroxy group-containing compound as a substrate compound of the above-described reaction may be added to the reaction mixture after the carbonyl halide-containing gas is blown into the composition containing the amide compound, or added to the reaction mixture before or while the carbonyl halide-containing gas is blown into the composition containing the amide compound.

The usage amount of the activating group-containing aromatic compound, the carboxylic acid compound and the hydroxy group-containing compound may be appropriately adjusted, and may be adjusted to, for example, 0.1 times or more by mole and 1.0 time or less by mole to the amide compound.

A Vilsmeier reagent is also useful for producing a carboxylic acid halide from a carboxylic acid compound. A Vilsmeier reagent reverts to an amide compound after the Vilsmeier reagent halogenates a carboxylic acid compound. An ester compound can be produced by reacting the produced carboxylic acid halide with an alcohol compound, and a carboxylic anhydride can be produced by reacting the produced carboxylic acid halide with a carboxylic acid. When a carboxylic acid compound and a base are used in place of the amide compound, the carboxylic acid compound may be anionized by the base and then the anionized carboxylate compound may be directly transformed to a carboxylic acid halide by the carbonyl halide. Such a carboxylic acid halide can be also used for producing an ester compound and a carboxylic anhydride.

8. Post-Treatment Step

Since many carbonyl halides are toxic, it is preferred that the produced carbonyl halide is not leaked out of the reaction system. For example, it is preferred that the gas phase discharged from the reaction vessel for the reaction of the produced carbonyl halide is supplied into an alcohol trap, and the gas phase discharged from the alcohol trap is further supplied into an alkali trap as demonstrated in FIGS. 1 to 4. The alcohol trap may be cooled as long as the alcohol to be used is not coagulated. For example, the alcohol trap may be cooled to −80° C. or higher and 50° C. or lower. In addition, for example, a sodium hydroxide aqueous solution and a saturated sodium hydrogencarbonate aqueous solution may be used for the alkali trap.

When the compound produced from the carbonyl halide is relatively unstable compound such as an isocyanate compound, further a reactive substrate compound may be added to the reaction mixture after the carbonyl halide is reacted. When the compound produced from the carbonyl halide is relatively stable compound such as a carbonate compound, the target compound may be purified from the reaction mixture. For example, water and a water-insoluble organic solvent such as chloroform are added to the reaction mixture, the aqueous phase and the organic phase are separated, the organic phase is dried over anhydrous sodium sulfate or anhydrous magnesium sulfate and then concentrated under reduced pressure, and the target compound may be purified by chromatography or the like.

The present application claims the benefit of the priority dates of Japanese patent application No. 2021-21002 filed on Feb. 12, 2021, and PCT application No. PCT/JP2022/002662 filed on Jan. 25, 2022. All of the contents of the Japanese patent application No. 2021-21002 filed on Feb. 12, 2021, and PCT application No. PCT/JP2022/002662 filed on Jan. 25, 2022 are incorporated by reference herein.

### EXAMPLES

Hereinafter, the examples are described to demonstrate the present invention more specifically, but the present invention is in no way restricted by the examples, and the examples can be appropriately modified to be carried out within a range that adapts to the contents of this specification. Such a modified example is also included in the range of the present invention.

### Example 1: Production of Phosgene

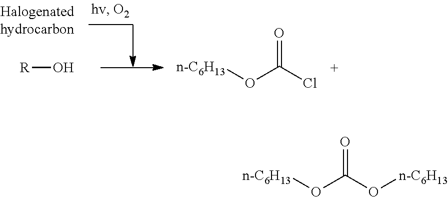

A reaction system was constructed by inserting a quartz glass jacket having the diameter of 30 mm in a cylindrical reaction vessel having the diameter of 42 mm and the volume of 100 mL and further inserting a low pressure mercury lamp (“UVL20PH-6” manufactured by SEN Light, 20 W, φ24 mm×120 mm) in the quartz glass jacket as schematically demonstrated in FIG. 1. The irradiated light contained UV-C having the wavelength of 185 nm and the wavelength of 254 nm. The illumination intensity of the light having the wavelength of 185 nm at the position 5 mm away from the center of the lamp to the reaction mixture was 2.00 to 2.81 mW/cm², and the illumination intensity of the light having the wavelength of 254 nm was 5.60 to 8.09 mW/cm². The cylindrical reaction vessel 2 was equipped with the cooling condenser 5-1 that was used for selectively transporting the produced low-boiling point gas component and cooled to 10° C., and a two-neck round bottom flask as the reaction vessel 6-1 equipped with the cooling condenser 5-2 cooled to −10° C. was connected thereto. The cooling condenser 5-2 was further connected to the two-neck round bottom flask containing an alcohol as the reaction vessel 6-2 and the trap vessel containing an alkali aqueous solution.

The bath temperature of the photoreaction vessel was adjusted to the temperature shown in Table 1, and then liquid halogenated hydrocarbon was supplied to the photoreaction vessel 2 from the PTFE tube having the inner diameter of 1 mm at the flow rate shown in Table 1 using a syringe pump. The halogenated hydrocarbon was stirred to accelerate vaporization. Next, oxygen was supplied to the gas phase of the reaction vessel 2 at the rate of 0.1 mL/min using the mass flow controller 7 to prepare a mixed gas of the halogenated hydrocarbon and oxygen in the vessel, and light was irradiated thereto using the low pressure mercury lamp. The gas produced by the oxidative photodecomposition of the mixed gas was blown into the stirred 1-hexanol (30 mL, 239 mmol) in the connected two-neck round bottom flask as the reaction vessel 6-1 at room temperature. The unreacted gas was trapped by the further connected 1-hexanol trap as the reaction vessel 6-2, and the waste gas from the reaction vessel 6-2 was treated by an alkali trap so that toxic gas was not leaked outside.

The yields of the chloroformic acid ester and the carbonate produced in the reaction vessel 6-1 and the reaction vessel 6-2 were estimated by ¹H NMR spectra, and the amount of the produced phosgene was determined on the basis of the total amounts thereof. The result is shown in Table 1.

TABLE 1

| Injected halogenated | | | | | | | Phosgene |
|---|---|---|---|---|---|---|---|
| hydrocarbon | | | Mixed gas | | | | yieldª |
| Halogenated | | Flow | Linear | Residence | Bath | Condenser | [Phosgene |
| hydrocarbon | Amount | rate | velocity | time | temp. | temp. | amount] |
| Cl₂C═CCl₂ | 10 mL | 4.0 mL/h | 0.342 m/min | 30 s | 140° C. | rt | 50% |
| | (98 mmol) | | | | | | [48.3 mmol] |
| C1₂C═CCl₂ | 10 mL | 4.0 mL/h | 0.360 m/min | 28 s | 160° C. | rt | 64% |
| | (98 mmol) | | | | | | [62.5 mmol] |
| Cl₂C═CC1₂ | 5 mL | 2.5 mL/h | 0.342 m/min | 30 s | 160° C. | 10° C. | 69% |
| | (49 mmol) | | | | | | [35.8 mmol] |
| Cl₂C═CC1₂ | 5 mL | 1.0 mL/h | 0.360 m/min | 28 s | 180° C. | 10° C. | 71% |
| | (49 mmol) | | | | | | [34.5 mmol] |
| Cl₂C═CC1₂ | 5 mL | 1.0 mL/h | 0.360 m/min | 28 s | 180° C. | 10° C. | 67% |
| | (49 mmol) | | | | | | [33.0 mmol] |

^{a}yield of phosgene to injected halogenated hydrocarbon

The halogenated hydrocarbon might be relatively rapidly decomposed to produce phosgene by continuously injecting the halogenated hydrocarbon from the outside using the syringe pump to the photoreaction vessel heated to the temperature of the boiling point of the halogenated hydrocarbon or higher for the gas phase photoreaction of the vaporized halogenated hydrocarbon and oxygen gas. The temperature of the reaction was increased to completely null the remaining liquid in the photoreaction vessel; as a result, phosgene could be produced at 180° C. with the yield of about 70% and the remaining liquid was not observed in the photoreaction vessel.

The produced phosgene gas did not contain notable by product and could be reacted with an alcohol to produce a chloroformic acid ester and a carbonate with high purity.

### Example 2: Synthesis of Chloroformic Acid Ester or Carbonate

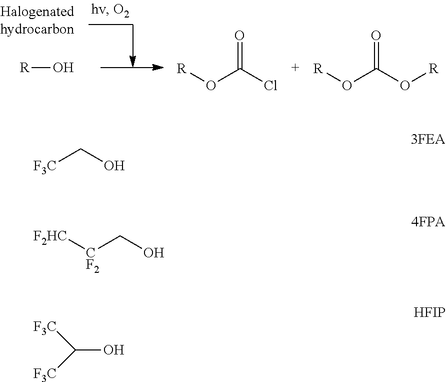

The temperature of the bath 3 was adjusted to 140° C. and the temperature of the cooling condenser 5-1 was adjusted to 20° C. in the reaction system demonstrated in FIG. 1. Oxygen was blown into the gas phase in the photoreaction vessel 2 at the rate of 0.1 mL/min using the mass flow controller 7 and light was irradiated from the low pressure mercury lamp for 3.5 hours or 6 hours while tetrachloroethylene was heated and refluxed in the photoreaction vessel 2.

The gas produced by the oxidative photodecomposition of the mixed gas was blown into the stirred dichloromethane solution of the reactive substrate alcohol or the stirred dichloromethane solution of the reactive substrate alcohol and the base in the connected two-neck round bottom flask as the reaction vessel 6-1 at room temperature. The unreacted gas was trapped by the further connected 1-butanol trap (10 mL) as the reaction vessel 6-2, and the gas discharged from the reaction vessel 6-2 was supplied into an alkali trap to be treated.

The yields of the chloroformic acid ester and the carbonate produced in the reaction vessel 6-1 and the reaction vessel 6-2 were determined by ¹H NMR spectra using 1,1,2,2-tetrachloroethane as an internal standard substance, and the amount of the produced phosgene was estimated from the total thereof.

In addition, the photoreaction was carried out in the gas-liquid mixing system by changing the bath temperature from 140° C. to 20° C. and supplying liquid tetrachloroethylene into the photoreaction vessel for comparison. The result is shown in Table 2.

TABLE 2

| | | | | | | Phosgene | | |
|---|---|---|---|---|---|---|---|---|
| | | Mixed gas | | | Light | yieldª | Yield [Amount] | |
| | | Linear | Residence | | irradiation | [Phosgene | Chloroformate | |
| Cl₂C═CCl₂ | Alcohol | velocity | time | Base | time | amount] | ester | Carbonate |

Bath temp.: 140° C.

| | | | | | | | | |
|---|---|---|---|---|---|---|---|---|
| 10 mL | MeOH | 0.330 m/min | 31 s | — | 3.5 h | 88% | 1% | 84% |
| (98 mmol) | (400 mmol) | | | | | [86.0 mmol] | [1 mmol] | [82 mmol] |
| 5 mL | 1-BuOH | 0.312 m/min | 33 s | — | 3.5 h | 82% | 78% | 4% |
| (49 mmol) | (60 mmol) | | | | | [40.1 mmol] | [38 mmol] | [2.1 mmol] |
| 10 mL | 3FEA | 0.318 m/min | 32 s | Pyridine | 6 h | 42% | | 41% |
| (98 mmol) | (120 mmol) | | | (240 mmol) | | [41.2 mmol] | — | [40 mmol] |
| 5 mL | 4FPA | 0.312 m/min | 33 s | NMI | 3.5 h | 54% | | 54% |
| (49 mmol) | (60 mmol) | | | (90 mmol) | | [26.0 mmol] | — | [26 mmol] |
| 5 mL | HFIP | 0.312 m/min | 33 s | Pyridine | 3.5 h | 37% | | 37% |
| (49 mmol) | (60 mmol) | | | (120 mmol) | | [18.0 mmol] | — | [18 mmol] |
| 5 mL | HFIP | 0.312 m/min | 33 s | NMI | 3.5 h | 40% | | 40% |
| (49 mmol) | (60 mmol) | | | (90 mmol) | | [30.0 mmol] | — | [19 mmol] |
| 5 mL | PhOH | 0.312 m/min | 33 s | Pyridine | 3.5 h | 73% | | 60% |
| (49 mmol) | (60 mmol) | | | (120 mmol) | | [36.7 mmol] | — | [29 mmol] |
| 5 mL | tBuOH | 0.312 m/min | 33 s | NMI | 3.5 h | 75% | 45% | 31% |
| (49 mmol) | (60 mmol) | | | (90 mmol) | | [32.1 mmol] | [22 mmol] | [15 mmol] |

Bath temp.: 20° C.

| | | | | | | | | |
|---|---|---|---|---|---|---|---|---|
| 5 mL | 1-BuOH | 0.213 m/min | 48 s | — | 3.5 h | 37% | 35% | 2% |
| (49 mmol) | (60 mmol) | | | | | [18.2 mmol] | [17.3 mmol] | [0.9 mmol] |

^{a}yield of phosgene to injected tetrachloroethane

When the reaction proceeded even without using a base, a high phosgene yield was obtained to the used tetrachloroethylene and ethyl chloroformate or a carbonate could be successfully produced as the result shown in Table 2.

On the one hand, in particular, when a fluorinated alcohol was used, the reaction tended to be difficult to proceed and thus a base was needed. When a base was not used and thus the reaction did not proceed, both of the phosgene yield and the carbonate yield were relatively low. The reason may be that even though phosgene was successfully produced by the photoreaction, the phosgene might be decomposed due to the base.

In addition, when the photoreaction was conducted in the gas-liquid mixing system by setting the bath temperature to 20° C. to directly supply liquid tetrachloroethylene to the photoreaction vessel as a comparative experiment with using a highly nucleophilic non-fluorinated alcohol as an alcohol and without using a base, which decomposes phosgene, the phosgene yield was relatively low and both of yields of the chloroformic acid ester and the carbonate were low.

It was found by the above results that phosgene can be efficiently produced by oxidatively photodecomposing a halogenated hydrocarbon in a gas phase.

In addition, dried air was used in place of oxygen, and the yield and yield amount of phosgene by the photoreaction in the conditions shown in Table 3 were calculated. The result is shown in Table 3.

TABLE 3

| | | | | | | Phosgene | | |
|---|---|---|---|---|---|---|---|---|
| | | Mixed gas | | | Light | yieldª | Yield [Amount] | |
| | | Linear | Residence | | irradiation | [Phosgene | Chloroformate | |
| Cl₂C═CCl₂ | Alcohol | velocity | time | Base | time | amount] | ester | Carbonate |

Bath temp.: 140° C.

| | | | | | | | | |
|---|---|---|---|---|---|---|---|---|
| 10 mL | 1-BuOH | 0.312 m/min | 33 s | — | 11 h | 43% | 31% | 12% |
| (98 mmol) | (200 mmol) | | | | | [42 mmol] | [30 mmol] | [12 mmol] |

^{a}yield of phosgene to injected tetrachloroethane

It was experimentally demonstrated by the result shown in Table 3 that even when dried air is used, the phosgene yield is relatively low but phosgene can be produced from tetrachloroethylene. The reason for the relatively low phosgene yield may be that the oxygen amount to tetrachloroethylene was decreased.

### Example 3: Synthesis of Hexyl Isocyanate by Supplying Gas Method

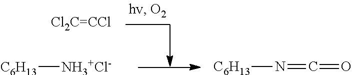

The temperature of the bath 3 was adjusted to 140° C., and the temperature of the cooling condenser 5-1 was adjusted to 20° C. Oxygen was supplied into the gas phase of the photoreaction vessel 2 at the velocity of 0.1 mL/min using the mass flow controller 7 with heating tetrachloroethylene (5 mL, 49 mmol) to reflux, and light was irradiated from the low pressure mercury lamp for 3.5 hours in the reaction system demonstrated in FIG. 1.

The gas produced by the oxidative photodecomposition of the mixed gas was blown into the stirred 1,1,2,2-tetrachloroethane solution (20 mL) of hexylamine hydrochloride salt (6.9 g, 50 mmol) in the connected two-neck round bottom flask as the reaction vessel 6 at 100° C.

Dichloromethane was added to the reaction mixture as an internal standard substance after the reaction, and the mixture was analyzed by ¹H NMR; as a result, it was confirmed that hexyl isocyanate was produced as the target compound (yield to tetrachloroethylene: 37%, yield to hexylamine hydrochloride salt: 37%).

### Example 4: Synthesis of Ethylene Carbonate by Supplying Gas Method

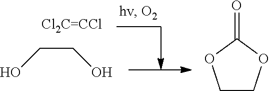

The temperature of the bath 3 was adjusted to 140° C., and the temperature of the cooling condenser 5-1 was adjusted to 20° C. Oxygen was supplied into the gas phase of the photoreaction vessel 2 at the velocity of 0.1 mL/min using the mass flow controller 7 with heating tetrachloroethylene (10 mL, 98 mmol) to reflux, and light was irradiated from the low pressure mercury lamp for 3.5 hours in the reaction system demonstrated in FIG. 1.

The gas produced by the oxidative photodecomposition of the mixed gas was blown into the stirred ethylene glycol (5.6 mL, 100 mmol) in the connected two-neck round bottom flask as the reaction vessel 6 at 0° C.

Dichloromethane was added to the reaction mixture as an internal standard substance after the reaction, and the mixture was analyzed by ¹H NMR; as a result, it was confirmed that ethylene carbonate was produced as the target compound (yield to tetrachloroethylene: 58%).

### Example 5: Synthesis of Amino Acid-N-Carboxylic Anhydride by Gas-Sending Method

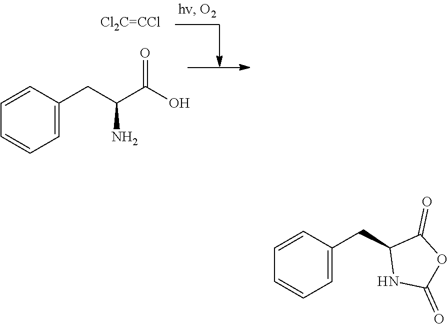

The temperature of the bath 3 was adjusted to 140° C., and the temperature of the cooling condenser 5-1 was adjusted to 20° C. Oxygen was supplied into the gas phase of the photoreaction vessel 2 at the velocity of 0.1 mL/min using the mass flow controller 7 with heating tetrachloroethylene (10 mL, 98 mmol) to reflux, and light was irradiated from the low pressure mercury lamp for 3.5 hours in the reaction system demonstrated in FIG. 1.

The gas produced by the oxidative photodecomposition of the mixed gas was blown into the stirred mixed solution containing L-phenylalanine (0.41 g, 2.5 mmol), chloroform (20 mL) and acetonitrile (20 mL) at 70° C. in the connected two-neck round bottom flask as the reaction vessel 6 equipped with the cooling condenser 5-2 of 0° C. The lamp was turned off, and then the reaction mixture was further stirred at 70° C. for 1 hour.

Dichloromethane was added to the reaction mixture as an internal standard substance after the reaction, and the mixture was analyzed by ¹H NMR; as a result, it was confirmed that the amino acid-N-carboxylic anhydride (NCA) was produced as the target compound (yield to N-phenylalanine: 60%).

### Example 6: Synthesis of Diphenylurea by Gas-Sending Method

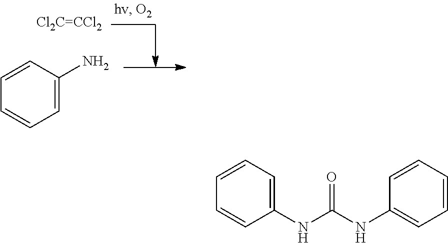

The temperature of the bath 3 was adjusted to 140° C., and the temperature of the cooling condenser 5-1 was adjusted to 20° C. Oxygen was supplied into the gas phase of the photoreaction vessel 2 at the velocity of 0.1 mL/min using the mass flow controller 7 with heating tetrachloroethylene (5 mL, 49 mmol) to reflux, and light was irradiated from the low pressure mercury lamp for 3.5 hours in the reaction system demonstrated in FIG. 1.

The gas produced by the oxidative photodecomposition of the mixed gas was blown into the stirred dichloromethane solution (50 mL) of aniline (42.3 g, 460 mmol) at 100° C. in the connected two-neck round bottom flask as the reaction vessel 6.

Water was added to the reaction mixture after the reaction, and the generated precipitate was washed and then separated by filtration. The precipitate was dried in vacuo at 50° C. for 3 hours as the white target compound (yield to tetrachloroethylene: 31%).

### Example 7: Synthesis of Polycarbonate by Gas-Sending Method

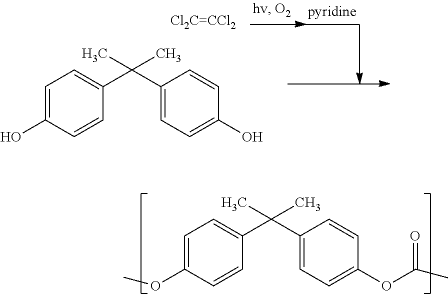

The temperature of the bath 3 was adjusted to 140° C., and the temperature of the cooling condenser 5-1 was adjusted to 20° C. Oxygen was supplied into the gas phase of the photoreaction vessel 2 at the velocity of 0.1 mL/min using the mass flow controller 7 with heating tetrachloroethylene (5 mL, 49 mmol) to reflux, and light was irradiated from the low pressure mercury lamp for 3.5 hours in the reaction system demonstrated in FIG. 1.

The gas produced by the oxidative photodecomposition of the mixed gas was blown into the stirred solution prepared by mixing Bisphenol A (4.5 g, 20 mmol), pyridine (4.0 mL, 50 mmol) and dichloromethane at room temperature in the connected two-neck round bottom flask as the reaction vessel 6.

Methanol (20 mL) was added to the reaction mixture after the reaction, and the generated precipitate was obtained by filtration and dried in vacuo as the white target compound (yield to tetrachloroethylene: 60%, yield to Bisphenol A: 95%).

The produced polycarbonate was analyzed by gel permeation chromatography (GPC) to determine the molecular weight. The result is shown in Table 4.

TABLE 4

| Mw | Mn | Mw/Mn |
|---|---|---|
| 3,980 | 2,230 | 1.78 |

### Example 8: Synthesis of Vilsmeier Reagent and Acid Chloride by Gas Phase Photoreaction

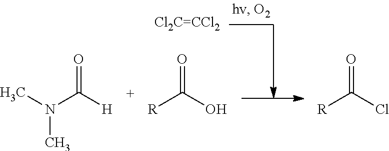

The temperature of the bath 3 was adjusted to 140° C., and the temperature of the cooling condenser 5-1 was adjusted to 20° C. Oxygen was supplied into the gas phase of the photoreaction vessel 2 at the velocity of 0.1 mL/min using the mass flow controller 7 with heating tetrachloroethylene (10 mL, 98 mmol) to reflux, and light was irradiated from the low pressure mercury lamp for 3.5 hours in the reaction system demonstrated in FIG. 1.

The gas produced by the oxidative photodecomposition of the mixed gas was blown into the stirred chloroform solution (100 mL) prepared by dissolving benzoic acid or propionic acid (200 mmol) and DMF (100 mmol) at 30° C. through the cooling condenser 5-1 adjusted to 20° C. in the connected two-neck round bottom flask as the reaction vessel 6 for 3.5 hours. The unreacted gas was supplied to the further connected alcohol trap and the alkali trap in order not to be leaked outside.

The reaction mixture was analyzed by ¹H NMR after the reaction; as a result, it was confirmed that the target carboxylic acid chloride was respectively produced with the yields of 54% and 35% to the used TCE. The result is shown in Table 5.

TABLE 5

| | | | Mixed gas | | Substrate | | |
|---|---|---|---|---|---|---|---|
| | Flow rate | | Linear | Residence | | Carboxylic | Yield |
| Cl₂C═CCl₂ | Cl₂C═CCl₂ | O₂ | velocity | time | DMF | acid | (Amount) |
| 98 mmol | 15.8 mL/min | 141 mL/min | 0.306 m/min | 31 s | 100 mmol | Benzoic | 54% |
| (10 mL) | (0.23 mmol/min) | (4.16 mmol/min) | | | | acid | (53.0 mmol) |
| | | | | | | 200 mmol | |
| 98 mmol | 15.8 mL/min | 141 mL/min | 0.306 m/min | 31 s | 100 mmol | Propionic | 35% |
| (10 mL) | (0.23 mmol/min) | (4.16 mmol/min) | | | | acid | (34.6 mmol) |
| | | | | | | 200 mmol | |

### Example 9: Synthesis of Vilsmeier Reagent by Gas Phase Photoreaction and Formylation Reaction

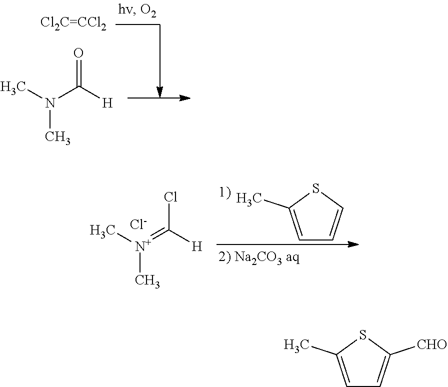

Tetrachloroethylene (5 mL, 49 mmol) was stirred in the photoreaction vessel 2 of the reaction system demonstrated in FIG. 1, and the temperature of the bath was increased to 140° C. to vaporize the tetrachloroethylene. Light was irradiated from the low pressure mercury lamp for 3.5 hours with supplying oxygen into the gas phase of the photoreaction vessel 2 at the velocity of 0.1 mL/min using the mass flow controller 7. The gas produced by the oxidative photodecomposition of the mixed gas was blown into the stirred DMF (3.9 mL, 50 mmol) at 30° C. through the cooling condenser 5-1 adjusted to 20° C. in the connected two-neck round bottom flask as the reaction vessel 6 for 3.5 hours. The unreacted gas was supplied to the further connected alcohol trap and the alkali trap in order not to be leaked outside.

Then, 2-methylthiophene (4.8 mL, 50 mmol) was added, and the mixture was stirred at 70° C. for 1 hour. Next, saturated sodium carbonate aqueous solution (30 mL) was added thereto for hydrolysis. Water and dichloromethane were further added, and the organic phase and the aqueous phase were separated. The organic phase was dried over anhydrous sodium sulfate, and the mixture was filtrated. The solvent was distilled away from the filtrate. The obtained dark green oil was analyzed by ¹H NMR; as a result; it was confirmed that the target aldehyde compound was produced with the yield of 30% to the used 2-methylthiophene. The result is shown in Table 6.

TABLE 6

| | | | Mixed gas | | O₂ | | Substrate | | |
|---|---|---|---|---|---|---|---|---|---|
| | Flow rate | | Linear | Residence | rate | Bath | | Aromatic | Yield |
| Cl₂C═CCl₂ | Cl₂C═CCl₂ | O₂ | velocity | time | flow | temp. | DMF | compound | (Amount) |
| 49 mmol | 7.9 mL/min | 141 mL/min | 0.306 m/min | 33 s | 0.1 L/min | 140° C. | 50 mmol | 2-Methyl | 30% |
| (5 mL) | (0.23 mmol/min) | (4.16 mmol/min) | | | | | | thiophene | (15 mmol) |
| | | | | | | | | 200 mmol | |

### EXPLANATION OF REFERENCES

1: Light source, 2: Photoreaction vessel, 3: Bath
4: Stirring bar, 5: Cooling condenser, 6: Reaction vessel,
7: Mass flow controller, 11: Syringe pump,
12: Mass flow controller, 13: Heater,
14: Flow photoreaction vessel, 15: Back pressure regulator,
16: Reaction vessel, 17: Trap vessel,
18: Syringe pump for injecting reaction substrate
19: Coil reaction deice, 20: Recovery vessel,
21: Tube reactor

1. A method for producing a carbonyl halide, comprising the steps of:
preparing a mixed gas comprising oxygen and a C₂₋₄ halogenated hydrocarbon having one or more halogeno groups selected from the group consisting of chloro, bromo and iodo, and
flowing the mixed gas and irradiating a high energy light to the flowed mixed gas.

2. The method according to claim 1, wherein a shortest distance from a light source of the high energy light to the flowed mixed gas is 1 m or less.

3. The method according to claim 1, wherein time to irradiate the high energy light to the flowed mixed gas is 1 second or more and 10000 seconds or less.

4. The method according to claim 1, wherein a temperature during the irradiation of the high energy light to the flowed mixed gas is 120° C. or higher and 300° C. or lower.

5. A method for producing a carbonate compound, comprising the steps of:
producing a halogenated carbony by the method according to claim 1, and
reacting an alcohol compound and the carbonyl halide,
wherein a molar ratio of the alcohol compound to the C₂₋₄ halogenated hydrocarbon is adjusted to 1 or more.

6. A method for producing a halogenated formic acid ester compound, comprising the steps of:
producing a halogenated carbony by the method according to claim 1, and
reacting an alcohol compound and the carbonyl halide,
wherein a molar ratio of the alcohol compound to the C₂₋₄ halogenated hydrocarbon is adjusted to less than 1.

7. A method for producing an isocyanate compound, comprising the steps of:
producing a halogenated carbony by the method according to claim 1, and
reacting a primary amine compound and the carbonyl halide,
wherein a molar ratio of the primary amine compound to the C₂₋₄ halogenated hydrocarbon is adjusted to less than 1.

8. A method for producing an amino acid-N-carboxylic anhydride, comprising the steps of:
producing a halogenated carbony by the method according to claim 1, and
reacting an amino acid compound represented by the following formula (VII) and the carbonyl halide,
wherein the amino acid-N-carboxylic anhydride is represented by the following formula (VIII): 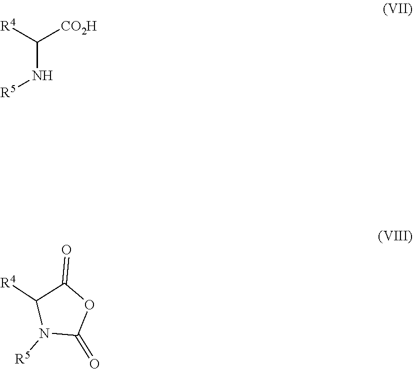
wherein
R⁴ is an amino acid side chain group wherein a reactive group is protected,
R⁵ is H or P¹—[—NH—CHR⁶—C(═O)—]₁— wherein R⁶ is an amino acid side chain group wherein a reactive group is protected, P¹ is a protective group of the amino group, l is an integer of 1 or more, and when l is an integer of 2 or more, a plurality of R⁶ may be the same as or different from each other.

9. A method for producing a Vilsmeier reagent,
wherein the Vilsmeier reagent is a salt represented by the following formula (X): 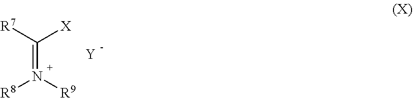
wherein
R⁷ is a hydrogen atom, a C₁₋₆ alkyl group or an optionally substituted C₆₋₁₂ aromatic hydrocarbon group,
R⁸ and R⁹ are independently a C₁₋₆ alkyl group or an optionally substituted C₆₋₁₂ aromatic hydrocarbon group, or R⁸ and R⁹ may form a 4 or more and 7 or less-membered ring structure together with each other,
X is a halogeno group selected from the group consisting of chloro, bromo and iodo,
Y⁻ is a counter anion,
comprising the steps of:
producing a halogenated carbony by the method according to claim 1, and
reacting the carbonyl halide and an amino acid compound represented by the following formula (IX): 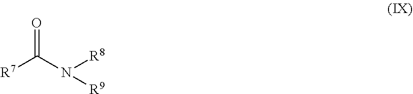
wherein R⁷ to R⁹ have the same meanings as the above.
